(19) United States
(12) Patent Application Publication
(10) Pub. No.: US 2011/0195989 A1
(43) Pub. Date: Aug. 11, 2011

(54) Controlled Release Formulations of Opioids

(76) Inventors: Edward M. Rudnic, Potomac, MA (US); Michael Vachon, Quebec (CA); Gary W. Pace, La Jolla, CA (US); Joseph Berry, Neshanic Station, NJ (US); Felix de la Iglesia, Ann Arbor, MI (US)

(21) Appl. No.: 13/024,319

(22) Filed: Feb. 9, 2011

Related U.S. Application Data
(60) Provisional application No. 61/302,698, filed on Feb. 9, 2010.
(60) Provisional application No. 61/386,277, filed on Sep. 24, 2010.

(51) Int. Cl. A61K 31/485; A61P 25/04

(52) U.S. Cl. 514/282

(57) ABSTRACT

Pharmaceutical formulations containing opioid components that each has a release profile. The components may provide immediate or controlled release of the opioid. The invention is also directed to methods of controlling release of one or more opioid compounds and methods of treating pain.

### CROSS-REFERENCES TO RELATED APPLICATIONS

This application claims priority to U.S. provisional application Ser. No. 61/302,698, filed Feb. 9, 2010, and to U.S. provisional application Ser. No. 61/386,277, filed Sep. 24, 2010, the entirety of both which is incorporated herein by reference.

### FIELD OF THE INVENTION

The invention is directed to pharmaceutical formulations comprising opioid components that each has a release profile. The components may provide immediate or controlled release of the opioid. The invention is also directed to methods of controlling release of one or more opioid compounds and methods of treating pain.

### BACKGROUND OF THE INVENTION

Opioids are a class of pain-relieving prescription medications frequently used in the treatment of a variety of acute and chronic, moderate to severe, pain. However, opioids can be rapidly absorbed and systemically excreted by the body through metabolic inactivation. In order to treat patients, especially those in severe pain, administration of opioids often requires careful dosing at frequent intervals to maintain effective steady state blood levels of the opioid, and thereby provide consistent analgesia. Otherwise, blood levels of the opioid can oscillate, resulting in poor and inconsistent pain relief.

These difficulties associated with the administration of opioids suggests a need to develop an opioid therapy that can, following administration, maintain consistent levels of opioid in the blood and avoid oscillations in pain relief.

### SUMMARY OF THE INVENTION

The invention relates to pharmaceutical formulations for treating pain that comprise components containing opioid compounds and having different release profiles. The invention also relates to methods of controlling release of one or more opioid compounds and methods of treating pain.

The pharmaceutical formulations of the invention may comprise one or more components having one or more release profiles, in which at least one of the components comprise a compound having opioid receptor agonist activity. In embodiments wherein there is more than one component, the components may have the same release profile, or the components may have different release profiles.

In some embodiments, the compounds having opioid receptor agonist activity may have agonist activity toward the mu (“μ,” morphine receptor), sigma (“σ,” the phencyclidine receptor), kappa (“κ,” the ketocyclazocine receptor) or delta (“δ,” the endorphinlenkephalin receptor) opioid receptors. Such compounds may include, among others, morphine, codeine, hydromorphone, hydrocodone, oxycodone, dihydrocodeine, dihydromorphine, oxymorphone, mixtures thereof, or salts thereof. In certain embodiments, a component may comprise two opioid compounds in varying ratios. In particular embodiments, a component may comprise morphine and oxycodone, or salts thereof, in about a 3:2 ratio by weight.

In some embodiments, the components may have an immediate release profile or a controlled release profile.

In certain embodiments, the formulation may comprise one or more additional components, such as at least two, at least three, at least four, or at least five components. In some embodiments, the one or more additional components may comprise one or more active agents. In some embodiments, the one or more active agents may be compounds having opioid receptor agonist activity. In some embodiments, the one or more active agents may be one or more non-opioid analgesic compound(s), or a mixture of one or more non-opioid analgesic compound(s) and one or more compound(s) with opioid receptor agonist activity, or pharmaceutically acceptable salts, esters or prodrugs thereof. In certain embodiments, the one or more active agents may be one or more hybrid opioid compound(s), or a mixture of one or more hybrid opioid compound(s) and one or more compound(s) with opioid receptor agonist activity, or pharmaceutically acceptable salts, esters or prodrugs thereof.

In embodiments of the invention, the pharmaceutical formulation may comprise one or more opioid components, wherein at least one of the opioid components is a controlled release opioid component that comprises an opioid. In certain embodiments, the opioid is selected from the group consisting of morphine, codeine, hydromorphone, hydrocodone, oxycodone, dihydrocodeine, dihydromorphine, oxymorphone, mixtures thereof, and salts thereof. In particular embodiments, the opioid is oxycodone or a salt thereof.

In certain embodiments, the pharmaceutical formulation provides a time to maximum opioid plasma concentration (Tₘₐₓ) of about 4.5 to about 8 hours after repeated administration. In particular embodiments, Tₘₐₓ is about 5 to about 6 hours, or about 6 hours, after repeated administration.

In some embodiments, the controlled release component provides a time to minimum oxycodone plasma concentration (Tₘᵢₙ) of about 13 to about 16 hours after repeated administration. In particular embodiments, Tₘᵢₙ is about 14 hours after repeated administration. In some embodiments, the repeated administration is through steady-state conditions.

In some embodiments, dissolution of the pharmaceutical formulation releases about 0 to about 20% of the opioid after two hours, or releases about 15 to about 60% of the opioid after four hours, or releases about 25 to about 80% of the opioid after six hours, or releases about 35 to about 85% of the opioid after eight hours, or releases about 45 to about 95% of the opioid after ten hours, or releases about 60 to about 100% of the opioid after twelve hours, as measured in a USP type I apparatus at 37° C. in water at 50 rpm.

In certain embodiments, when the pharmaceutical formulation comprises about 2 mg of opioid, the pharmaceutical formulation may provide a mean maximum plasma concentration (Cₘₐₓ) of about 1 to about 3 ng/mL, or about 2 ng/mL, after repeated administration. In some embodiments, the repeated administration may be through steady-state conditions. In certain embodiments, the area-under-the-curve for between about 0 and about 24 hours (AUC₂₄) may be about 14.7 ng·hr/mL to about 23.0 ng·hr/mL, or about 15.8 ng·hr/mL to about 21.0 ng·hr/mL, or about 17.1 ng·hr/mL to about 19.7 ng·hr/mL, after single administration.

In certain embodiments, when the pharmaceutical formulation comprises about 5 mg of opioid, the pharmaceutical formulation may provide a mean Cₘₐₓ of about 3 to about 7 ng/mL, or about 5 ng/mL, after repeated administration. In some embodiments, the repeated administration may be through steady-state conditions. In certain embodiments, the AUC₂₄ may be about 40.2 ng·hr/mL to about 62.8 ng·hr/mL, or about 43.2 ng·hr/mL to about 57.2 ng·hr/mL, or about 46.7 ng·hr/mL to about 53.7 ng·hr/mL, after single administration.

In certain embodiments, when the pharmaceutical formulation comprises about 10 mg of opioid, the pharmaceutical formulation may provide a mean Cₘₐₓ of about 5 to about 15 ng/mL, or about 10 ng/mL, after repeated administration. In some embodiments, the repeated administration may be through steady-state conditions. In certain embodiments, the AUC₂₄ may be about 80.5 ng·hr/mL to about 125.9 ng·hr/mL, or about 86.6 ng·hr/mL to about 114.8 ng·hr/mL, or about 93.7 ng·hr/mL to about 107.7 ng·hr/mL, after single administration.

In certain embodiments, when the pharmaceutical formulation comprises about 20 mg of opioid, the pharmaceutical formulation may provide a mean Cₘₐₓ of about 10 to about 30 ng/mL, or about 20 ng/mL, after repeated administration. In some embodiments, the repeated administration may be through steady-state conditions. In certain embodiments, the AUC₂₄ may be about 166.0 ng·hr/mL to about 259.3 ng·hr/mL, or about 178.5 ng·hr/mL to about 236.6 ng·hr/mL, or about 193.0 ng·hr/mL to about 222.0 ng·hr/mL, after single administration.

In certain embodiments, when the pharmaceutical formulation comprises about 40 mg of opioid, the pharmaceutical formulation may provide a mean Cₘₐₓ of about 25 to about 55 ng/mL, or about 40 ng/mL, after repeated administration. In some embodiments, the repeated administration may be through steady-state conditions. In certain embodiments, the AUC₂₄ may be about 338.5 ng·hr/mL to about 528.9 ng·hr/mL, or about 363.9 ng·hr/mL to about 482.3 ng·hr/mL, or about 393.5 ng·hr/mL to about 452.7 ng·hr/mL, after single administration.

In certain embodiments, when the pharmaceutical formulation comprises about 80 mg of opioid, the pharmaceutical formulation may provide a mean Cₘₐₓ of about 50 to about 110 ng/mL, or about 80 ng/mL, after repeated administration. In some embodiments, the repeated administration may be through steady-state conditions. In certain embodiments, the AUC₂₄ may be about 868.4 ng·hr/mL to about 1356.9 ng·hr/mL, or about 933.5 ng·hr/mL to about 1237.5 ng·hr/mL, or about 1009.5 ng·hr/mL to about 1161.5 ng·hr/mL, after single administration.

In some embodiments, the pharmaceutical formulation provides a mean minimum oxycodone plasma concentration (Cₘᵢₙ) of about 0.5 to about 40 ng/mL, or about 4 to about 15 ng/mL, after repeated administration. In some embodiments, the repeated administration is through steady-state conditions.

In certain embodiments, the pharmaceutical formulation comprises a second controlled release opioid component. In some embodiments, the second controlled release opioid component comprises an opioid selected from the group consisting of morphine, codeine, hydromorphone, hydrocodone, oxycodone, dihydrocodeine, dihydromorphine, oxymorphone, mixtures thereof, and salts thereof.

In certain embodiments, the pharmaceutical formulation comprises an immediate-release opioid component. In some embodiments, the immediate-release opioid component comprises an opioid selected from the group consisting of morphine, codeine, hydromorphone, hydrocodone, oxycodone, dihydrocodeine, dihydromorphine, oxymorphone, mixtures thereof, and salts thereof. In further embodiments, the opioid in the immediate-release opioid component is morphine or a salt thereof. In yet further embodiments, the total morphine, or salt thereof, and the total oxycodone, or salt thereof, in the formulation are in a ratio of about 3:2, morphine or salt thereof to oxycodone or salt thereof, by weight.

In certain embodiments, the pharmaceutical formulation comprises a second opioid component and a third opioid component, wherein: (a) the second opioid component is an immediate-release opioid component and comprises an opioid having κ agonist activity; and (b) the third opioid component is a controlled release opioid component and comprises an opioid having mu agonist activity. In some embodiments, the opioid having κ agonist activity is oxycodone or a salt thereof, and the opioid having μ agonist activity is morphine or a salt thereof.

In certain embodiments, the controlled release opioid component comprises morphine or a salt thereof. In some embodiments, the controlled release opioid component comprises morphine or salt thereof and oxycodone or salt thereof in an amount of about 3:2 by weight.

In embodiments of the invention, the pharmaceutical formulation may comprise one or more opioid components for humans in need thereof, such that the one or more opioid components comprise one or more release profiles, and at least one of the opioid components is a controlled release opioid component comprising oxycodone or a salt thereof; pharmaceutical formulation, when containing a total dose of oxycodone, or a salt thereof, of about 2 mg, provides a Tₘₐₓ of about 4.5 to about 7.5 hours, or about 5 to about 6 hours, or about 6 hours, after repeated administration, and an AUC₂₄ of about 14.7 ng·hr/mL to about 23.0 ng·hr/mL, or about 15.8 ng·hr/mL to about 21.0 ng·hr/mL, or about 17.1 ng·hr/mL to about 19.7 ng·hr/mL, after single administration. The repeated administration may be through steady-state conditions.

In certain embodiments, the pharmaceutical formulation is formulated for a total dose of oxycodone, or a salt thereof, that is different from about 2 mg of oxycodone, or a salt thereof, and has an AUC₂₄ that is proportional to the 2 mg AUC₂₄. In some embodiments, the total dose AUC₂₄ is linearly proportional to the 2 mg AUC₂₄.

In certain embodiments, the pharmaceutical formulation, when containing a total dose of oxycodone, or a salt thereof, of about 2 mg, provides a Cₘₐₓ of about 1 to about 3 ng/mL, or about 2 mg, after repeated administration. In some embodiments, the repeated administration is through steady-state conditions. In some embodiments, the pharmaceutical formulation is formulated for a total dose of oxycodone, or a salt thereof, that is different from about 2 mg of oxycodone, or a salt thereof, and has a Cₘₐₓ that is proportional to the 2 mg Cₘₐₓ. In some embodiments, the total dose Cₘₐₓ is linearly proportional to the 2 mg Cₘₐₓ.

In certain embodiments, the pharmaceutical formulation, when containing a total dose of about 2 mg, provides a Tₘᵢₙ of about 13 to about 16 hours after repeated administration. In some embodiments, the repeated administration is through steady-state conditions.

In embodiments of the invention, the pharmaceutical formulation may comprise one or more opioid components for humans in need thereof, such that the one or more opioid components comprise one or more release profiles, and at least one of the opioid components is a controlled release opioid component comprising oxycodone or a salt thereof; the pharmaceutical formulation, when containing a total dose of oxycodone, or a salt thereof, of about 5 mg, provides a Tₘₐₓ of about 4.5 to about 7.5 hours, or about 5 to about 6 hours, or about 6 hours, after repeated administration, and an AUC₂₄ of about 40.2 ng·hr/mL to about 62.8 ng·hr/mL, or about 43.2 ng·hr/mL to about 57.2 ng·hr/mL, or about 46.7 ng·hr/mL to about 53.7 ng·hr/mL, after single administration. The repeated administration may be through steady-state conditions.

In certain embodiments, the pharmaceutical formulation is formulated for a total dose of oxycodone, or a salt thereof, that is different from about 5 mg of oxycodone, or a salt thereof, and has an AUC₂₄ that is proportional to the 5 mg AUC₂₄. In some embodiments, the total dose AUC₂₄ is linearly proportional to the 5 mg AUC₂₄.

In certain embodiments, the pharmaceutical formulation, when containing a total dose of oxycodone, or a salt thereof, of about 5 mg, provides a Cₘₐₓ of about 3 to about 7 ng/mL, or about 5 ng/mL, after repeated administration. In some embodiments, the repeated administration is through steady-state conditions. In some embodiments, the pharmaceutical formulation is formulated for a total dose of oxycodone, or a salt thereof, that is different from about 5 mg of oxycodone, or a salt thereof, and has a Cₘₐₓ that is proportional to the 5 mg Cₘₐₓ. In some embodiments, the total dose Cₘₐₓ is linearly proportional to the 5 mg Cₘₐₓ.

In certain embodiments, the pharmaceutical formulation, when containing a total dose of oxycodone, or a salt thereof, of about 5 mg, provides a Tₘᵢₙ of about 13 to about 16 hours after repeated administration. In some embodiments, the repeated administration is through steady-state conditions.

In embodiments of the invention, the pharmaceutical formulation may comprise one or more opioid components for humans in need thereof, such that the one or more opioid components comprise one or more release profiles, and at least one of the opioid components is a controlled release opioid component comprising oxycodone or a salt thereof; the pharmaceutical formulation, when containing a total dose of oxycodone, or a salt thereof, of about 10 mg, provides a Tₘₐₓ of about 4.5 to about 7.5 hours, or about 5 to about 6 hours, or about 6 hours, after repeated administration, and an AUC₂₄ of about 80.5 ng·hr/mL to about 125.9 ng·hr/mL, or about 86.6 ng·hr/mL to about 114.8 ng·hr/mL, or about 93.7 ng·hr/mL to about 107.7 ng·hr/mL, after single administration. The repeated administration may be through steady-state conditions.

In certain embodiments, the pharmaceutical formulation is formulated for a total dose of oxycodone, or a salt thereof, that is different from about 10 mg of oxycodone, or a salt thereof, and has an AUC₂₄ that is proportional to the 10 mg AUC₂₄. In some embodiments, the total dose AUC₂₄ is linearly proportional to the 10 mg AUC₂₄.

In certain embodiments, the pharmaceutical formulation, when containing a total dose of oxycodone, or a salt thereof, of about 10 mg, provides a Cₘₐₓ of about 5 to about 15 ng/mL, or about 10 ng/mL, after repeated administration. In some embodiments, the repeated administration is through steady-state conditions. In some embodiments, the pharmaceutical formulation is formulated for a total dose of oxycodone, or a salt thereof, that is different from about 10 mg of oxycodone, or a salt thereof, and has a Cₘₐₓ that is proportional to the 10 mg Cₘₐₓ. In some embodiments, the total dose Cₘₐₓ is linearly proportional to the 10 mg Cₘₐₓ.

In certain embodiments, the pharmaceutical formulation, when containing a total dose of oxycodone, or a salt thereof, of about 10 mg, provides a Tₘᵢₙ of about 13 to about 16 hours after repeated administration. In some embodiments, the repeated administration is through steady-state conditions.

In embodiments of the invention, the pharmaceutical formulation may comprise one or more opioid components for humans in need thereof, such that the one or more opioid components comprise one or more release profiles, and at least one of the opioid components is a controlled release opioid component comprising oxycodone or a salt thereof; the pharmaceutical formulation, when containing a total dose of oxycodone, or a salt thereof, of about 20 mg, provides a Tₘₐₓ of about 4.5 to about 7.5 hours, or about 5 to about 6 hours, or about 6 hours, after repeated administration, and an AUC₂₄ of about 166.0 ng·hr/mL to about 259.3 ng·hr/mL, or about 178.5 ng·hr/mL to about 236.6 ng·hr/mL, or about 193.0 ng·hr/mL to about 222.0 ng·hr/mL, after single administration. The repeated administration may be through steady-state conditions.

In certain embodiments, the pharmaceutical formulation is formulated for a total dose of oxycodone, or a salt thereof, that is different from about 20 mg of oxycodone, or a salt thereof, and has an AUC₂₄ that is proportional to the 20 mg AUC₂₄. In some embodiments, the total dose AUC₂₄ is linearly proportional to the 20 mg AUC₂₄.

In certain embodiments, the pharmaceutical formulation, when containing a total dose of oxycodone, or a salt thereof, of about 20 mg, provides a Cₘₐₓ of about 10 to about 30 ng/mL, or about 20 ng/mL, after repeated administration. In some embodiments, the repeated administration is through steady-state conditions. In some embodiments, the pharmaceutical formulation is formulated for a total dose of oxycodone, or a salt thereof, that is different from about 20 mg of oxycodone, or a salt thereof, and has a Cₘₐₓ that is proportional to the 20 mg Cₘₐₓ. In some embodiments, the total dose Cₘₐₓ is linearly proportional to the 20 mg Cₘₐₓ.

In certain embodiments, the pharmaceutical formulation, when containing a total dose of oxycodone, or a salt thereof, of about 20 mg, provides a Tₘᵢₙ of about 8 hours after repeated administration. In some embodiments, the repeated administration is through steady-state conditions. In some embodiments, the pharmaceutical formulation is formulated for a total dose different from about 20 mg of oxycodone, or a salt thereof, and having a Cₘᵢₙ proportional to the 20 mg Cₘᵢₙ. In some embodiments, the total dose Cₘᵢₙ is linearly proportional to the 20 mg Cₘᵢₙ.

In embodiments of the invention, the pharmaceutical formulation may comprise one or more opioid components for humans in need thereof, such that the one or more opioid components comprise one or more release profiles, and at least one of the opioid components is a controlled release opioid component comprising oxycodone or a salt thereof; the pharmaceutical formulation, when containing a total dose of oxycodone, or a salt thereof, of about 40 mg, provides a Tₘₐₓ of about 4.5 to about 7.5 hours, or about 5 to about 6 hours, or about 6 hours, after repeated administration, and an AUC₂₄ of about 338.5 ng·hr/mL to about 528.9 ng·hr/mL, or about 363.9 ng·hr/mL to about 482.3 ng·hr/mL, or about 393.5 ng·hr/mL to about 452.7 ng·hr/mL, after single administration. The repeated administration may be through steady-state conditions.

In certain embodiments, the pharmaceutical formulation is formulated for a total dose of oxycodone, or a salt thereof, that is different from about 40 mg of oxycodone, or a salt thereof, and has an AUC₂₄ that is proportional to the 40 mg AUC₂₄. In some embodiments, the total dose AUC₂₄ is linearly proportional to the 40 mg AUC₂₄.

In certain embodiments, the pharmaceutical formulation, when containing a total dose of oxycodone, or a salt thereof, of about 40 mg, provides a Cₘₐₓ of about 25 to about 55 ng/mL, or about 40 ng/mL, after repeated administration. In some embodiments, the repeated administration is through steady-state conditions. In some embodiments, the pharmaceutical formulation is formulated for a total dose of oxycodone, or a salt thereof, that is different from about 40 mg of oxycodone, or a salt thereof, and has a Cₘₐₓ that is proportional to the 40 mg Cₘₐₓ. In some embodiments, the total dose Cₘₐₓ is linearly proportional to the 40 mg Cₘₐₓ.

In certain embodiments, the pharmaceutical formulation, when containing a total dose of oxycodone, or a salt thereof, of about 40 mg, provides a Tₘᵢₙ of about 13 to about 16 hours after repeated administration. In some embodiments, the repeated administration is through steady-state conditions.

In embodiments of the invention, the pharmaceutical formulation may comprise one or more opioid components for humans in need thereof, such that the one or more opioid components comprise one or more release profiles, and at least one of the opioid components is a controlled release opioid component comprising oxycodone or a salt thereof; the pharmaceutical formulation, when containing a total dose of oxycodone, or a salt thereof, of about 80 mg, provides a Tₘₐₓ of about 4.5 to about 7.5 hours, or about 5 to about 6 hours, or about 6 hours, after repeated administration, and an AUC₂₄ of about 868.4 ng·hr/mL to about 1356.9 ng·hr/mL, or about 933.5 ng·hr/mL to about 1237.5 ng·hr/mL, or about 1009.5 ng·hr/mL to about 1161.5 ng·hr/mL, after single administration. The repeated administration may be through steady-state conditions.

In certain embodiments, the pharmaceutical formulation is formulated for a total dose of oxycodone, or a salt thereof, that is different from about 80 mg of oxycodone, or a salt thereof, and has an AUC₂₄ that is proportional to the 80 mg AUC₂₄. In some embodiments, the total dose AUC₂₄ is linearly proportional to the 80 mg AUC₂₄.

In certain embodiments, the pharmaceutical formulation, when containing a total dose of oxycodone, or a salt thereof, of about 80 mg, provides a Cₘₐₓ of about 50 to about 110 ng/mL, or about 80 ng/mL, after repeated administration. In some embodiments, the repeated administration is through steady-state conditions. In some embodiments, the pharmaceutical formulation is formulated for a total dose of oxycodone, or a salt thereof, that is different from about 80 mg of oxycodone, or a salt thereof, and has a Cₘₐₓ that is proportional to the 80 mg Cₘₐₓ. In some embodiments, the total dose Cₘₐₓ is linearly proportional to the 80 mg Cₘₐₓ.

In certain embodiments, the pharmaceutical formulation, when containing a total dose of oxycodone, or a salt thereof, of about 80 mg, provides a Tₘᵢₙ of about 13 to about 16 hours after repeated administration. In some embodiments, the repeated administration is through steady-state conditions.

The method for controlling release of one or more compounds having opioid receptor agonist activity for absorption in a human comprises administering a pharmaceutical formulation comprising one or more components, such that the one or more opioid components comprise one or more release profiles, and at least one of the opioid components is a controlled release opioid component comprising an opioid. In certain embodiments, the pharmaceutical formulation administered to the human is in accordance to the pharmaceutical formulations of the invention.

The method of treating pain in a human comprises administering a pharmaceutical formulation comprising one or more components, such that the one or more opioid components comprise one or more release profiles, and at least one of the opioid components is a controlled release opioid component comprising an opioid. In certain embodiments, the pharmaceutical formulation administered to the human is in accordance to the pharmaceutical formulations of the invention.

### BRIEF DESCRIPTION OF DRAWINGS

FIGS. 1a and 1b provide schematic images of two embodiments of opioid formulations of the present invention.

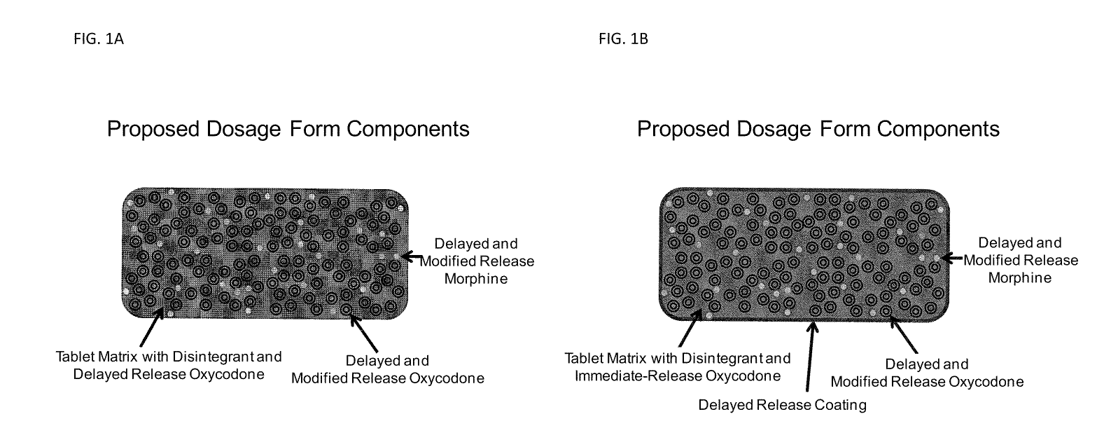

FIG. 2 provides a target release profile for oxycodone coated pellets used in the opioid formulations of the present invention.

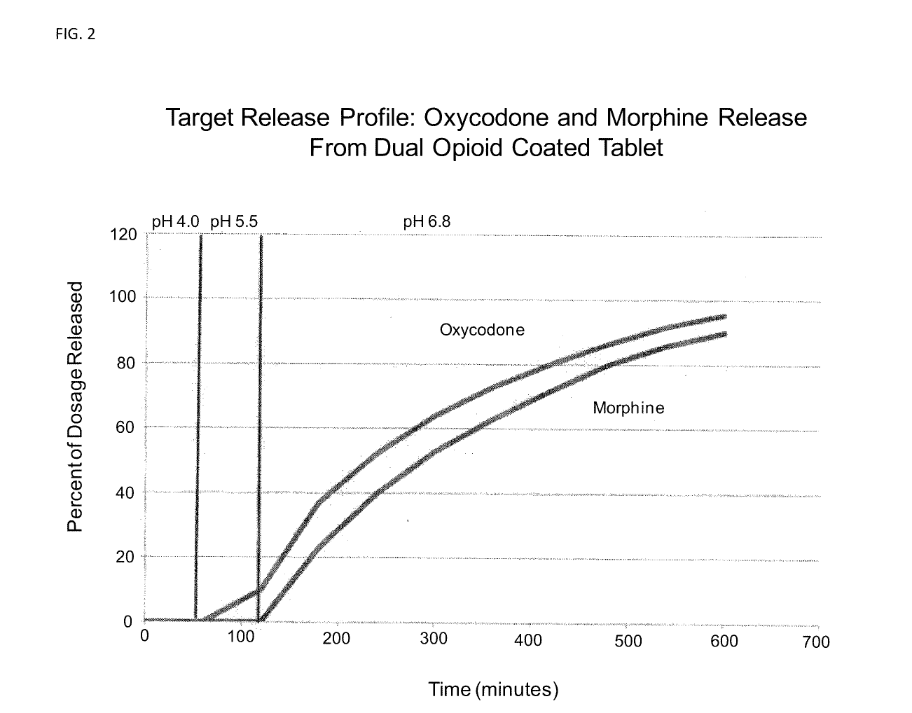

FIG. 3 provides a target release profile for morphine coated pellets used in the opioid formulations of the present invention.

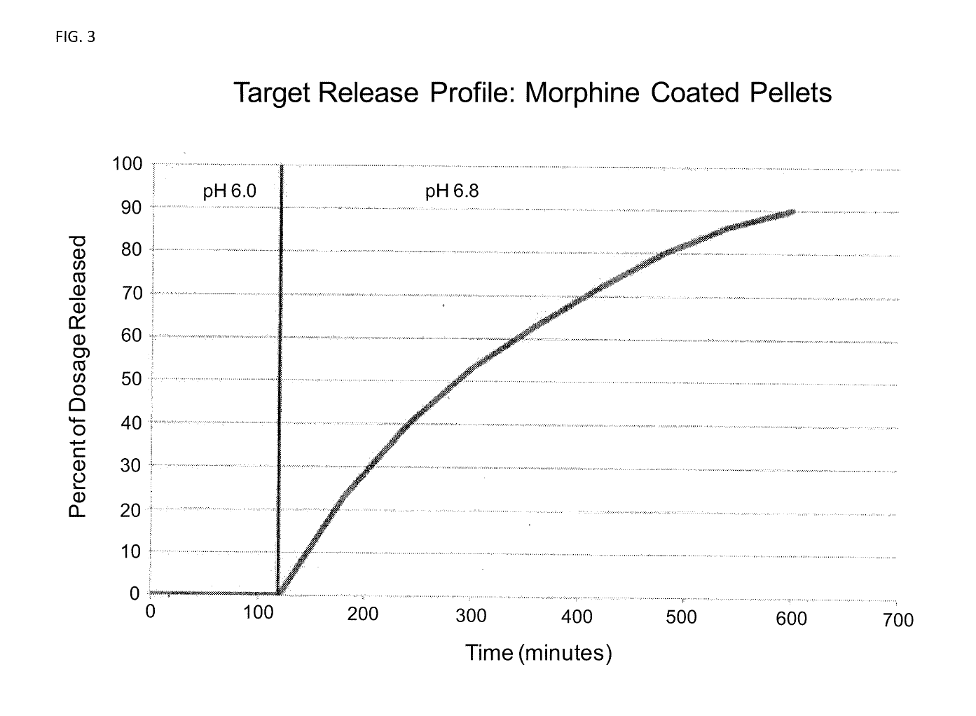

FIG. 4 provides a target release profile for oxycodone granulation coated only with Eudragit L30D-55 used in the opioid formulations of the present invention.

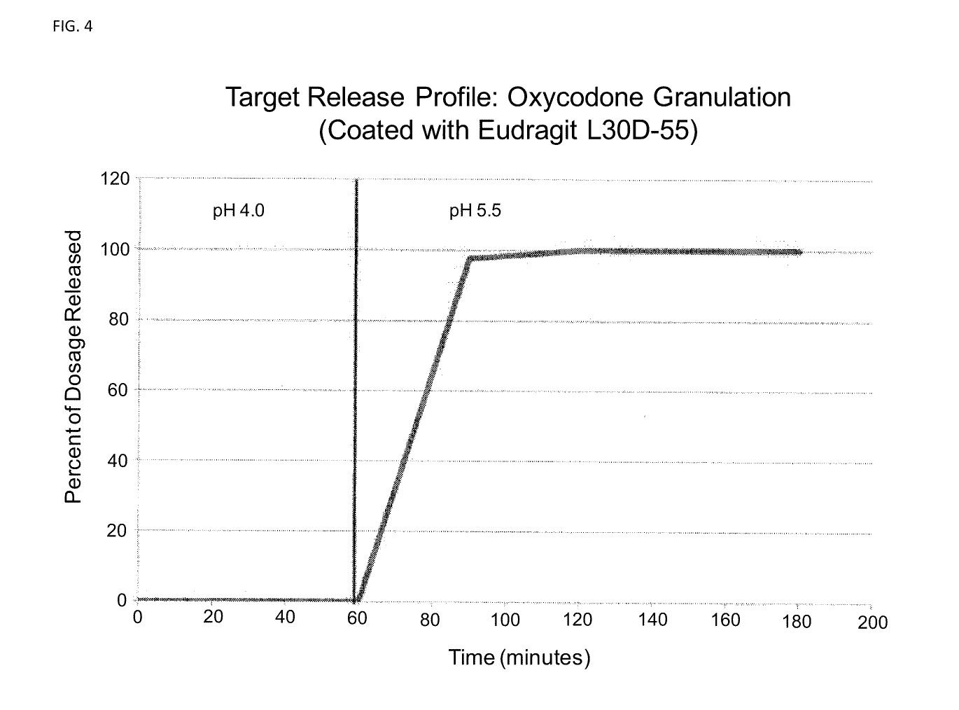

FIG. 5 provides a target release profile for total oxycodone release in the opioid formulations of the present invention.

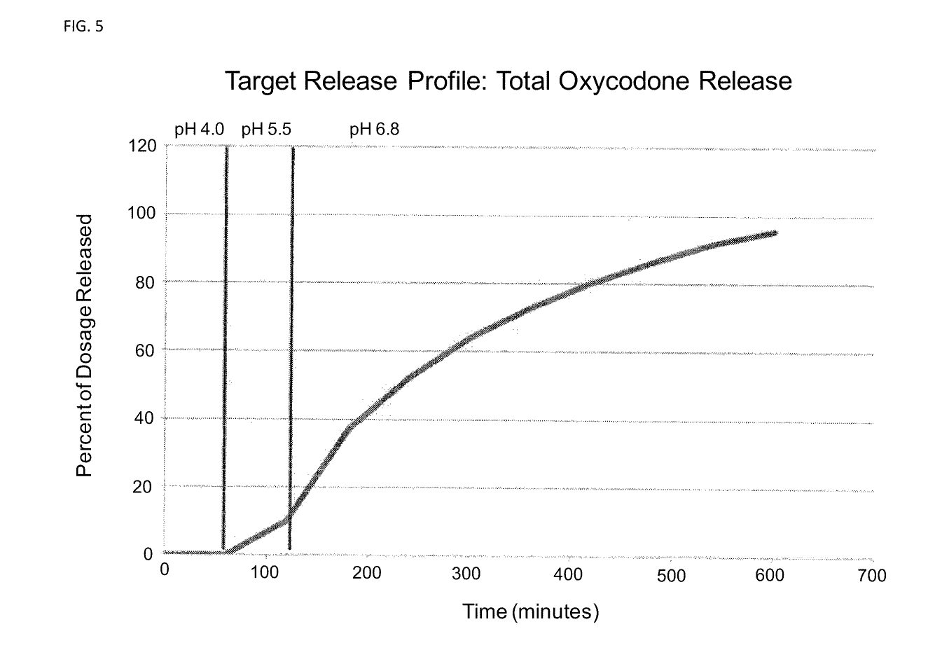

FIG. 6 provides a target release profile for total oxycodone and morphine release used in the dual-opioid coated tablets of the present invention.

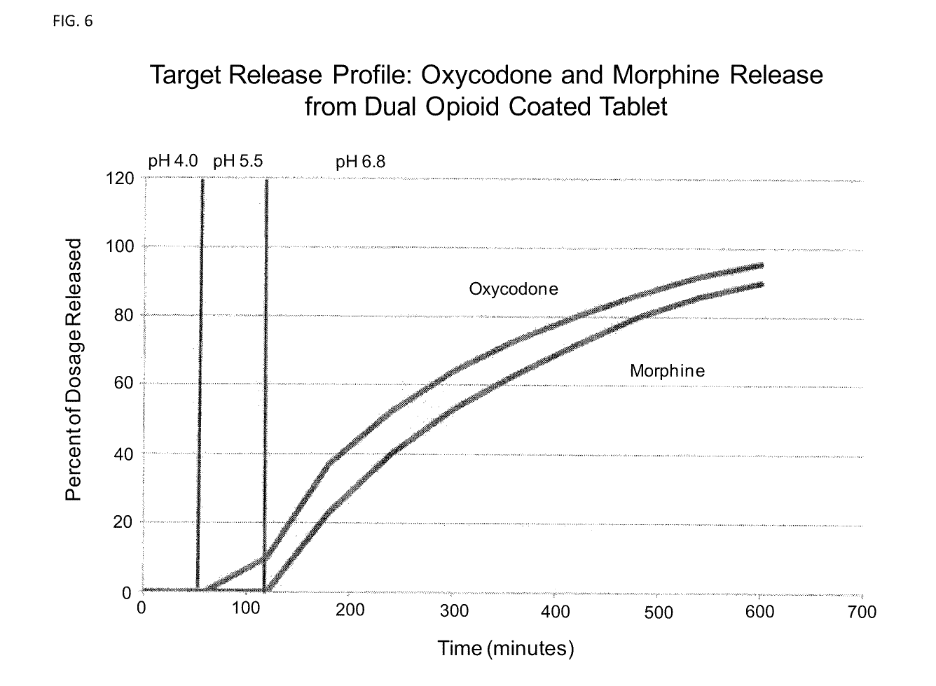

FIG. 7 provides a schematic demonstrating the methods used in producing the oxycodone granules used in the present invention.

FIG. 8 provides a schematic demonstrating the methods used in producing the oxycodone core pellets used in the present invention.

FIG. 9 provides a schematic demonstrating the methods used in producing the morphine core pellets used in the present invention.

FIG. 10 provides a schematic demonstrating the methods used in coating the either morphine or oxycodone core pellets used in the present invention.

FIG. 11 provides a schematic demonstrating the methods used in producing the dual opioid coated tablets used in the present invention.

FIG. 12 provides a flow diagram for preparing extended release intermediate oxycodone pellets used in the clinical study (Example 2).

FIG. 13 provides an oxycodone plasma concentration profile of two opioid formulations of the present invention (Formulation A and Formulation B) and a Reference Formulation (MS Contin® 30 mg (morphine CR) co-administered with OxyContin® 20 mg (oxycodone CR)) through 72 hours after treatment.

FIG. 14 provides an oxycodone plasma concentration profile of two opioid formulations of the present invention (Formulation A and Formulation B) and a Reference Formulation (MS Contin® 30 mg (morphine CR) co-administered with OxyContin® 20 mg (oxycodone CR)) through 24 hours after treatment.

FIG. 15 provides a projected oxycodone plasma profile from administration of multiple doses at 12 hour intervals of an opioid formulation of the present invention.

FIG. 16 provides a projected oxycodone plasma profile from administration of multiple doses of an opioid formulation of the present invention having different dosing strengths.

FIG. 17 provides a projected oxycodone plasma profile from administration of multiple doses at 12 hour intervals of an opioid composite formulation (immediate release+controlled release) of the present invention.

FIG. 18 provides a projected oxycodone plasma profile from administration of multiple doses of an opioid composite formulation (immediate release+controlled release) of the present invention having different dosing strengths.

FIG. 19 provides a release profile of morphine sulfate from coated beadlets containing morphine sulfate and oxycodone hydrochloride using Ammonio Methacrylate Copolymer Type B (RS) and Type A (RL) coating ratios of (a) RS/RL=90/10 and (b) RS/RL=80/20, at various % coating levels.

FIG. 20 provides a release profile of oxycodone hydrochloride from coated beadlets containing morphine sulfate and oxycodone hydrochloride using Ammonio Methacrylate Copolymer Type B (RS) and Type A (RL) coating ratios of (a) RS/RL=90/10 and (b) RS/RL=80/20, at various % coating levels.

FIG. 21 provides a release profile of morphine sulfate in enteric coated tablets (using 50% coated beadlets of Ammonio Methacrylate Copolymer Type B (RS) and Type A (RL) in a ratio of 90/10) at various % enteric coating levels.

FIG. 22 provides a release profile of oxycodone hydrochloride in enteric coated tablets (using 50% coated beadlets of Ammonio Methacrylate Copolymer Type B (RS) and Type A (RL) in a ratio of 90/10) at various % enteric coating levels.

FIG. 23 provides a release profile for morphine sulfate in enteric coated tablets (10% and 15% coating level) at low, mid or high hardness levels.

FIG. 24 provides a release profile for oxycodone hydrochloride in enteric coated tablets (10% and 15% coating level) at low, mid or high hardness levels.

FIG. 25 provides a release profile of oxycodone hydrochloride from coated beadlets containing oxycodone hydrochloride using Ammonio Methacrylate Copolymer Type B (RS) and Type A (RL) coating ratios of RS/RL=85/15 at various % coating levels.

FIG. 26 provides a release profile of oxycodone hydrochloride from coated beadlets containing oxycodone hydrochloride using Ammonio Methacrylate Copolymer Type B (RS) and Type A (RL) coating ratios of RS/RL=80/20 at various % coating levels.

FIG. 27 provides a release profile of oxycodone hydrochloride from coated beadlets containing oxycodone hydrochloride using Ammonio Methacrylate Copolymer Type B (RS) and Type A (RL) coating ratios of RS/RL=80/20 and RS/RL=85/15 at various % coating levels.

### DETAILED DESCRIPTION OF THE INVENTION

The invention relates to pharmaceutical formulations and methods for the alleviation of acute or chronic pain by controlling the release of compounds having opioid agonist activity for absorption in humans. The pharmaceutical formulations and methods of the invention may provide effective analgesia to a patient while reducing or eliminating undesired side effects typically experienced with the administration of opioid analgesic compounds. Due to the controlled release of the compound (s), it is possible to obtain a substantially constant rate of release of the compound(s) over a specific period of time, corresponding to the dosage necessary for the treatment in question, so that adherence to a strict dosage regimen, e.g. requiring administration of a drug at set intervals up to several times a day, may be dispensed with.

One aspect of the invention relates to pharmaceutical formulations comprising one or more components having one or more release profiles, such that at least one of the components comprises a compound having opioid receptor agonist activity and has a controlled release profile. Another aspect of the invention relates to the administration of the pharmaceutical formulations of the invention to humans in need thereof.

The formulations and methods described herein are used to treat different types of pain, including neuropathic pain and nociceptive pain, somatic pain and visceral pain. In various embodiments, formulations and methods described herein are used to treat diabetic neuropathy, trigeminal neuralgia, postherpetic zoster pain, and thalamic pain syndrome (a central pain). Neuropathic pain frequently coexists with nociceptive pain, and the inventive compounds and salts may be used to treat mixed pain states, i.e. a combination of neuropathic and nociceptive pain. For example, trauma that damages tissue and nerves, burns (that burn skin as well as nerve endings), and external nerve compression may cause both neuropathic and nociceptive pain. Examples of external nerve compression include tumor nerve compression and sciatica from herniated discs pressing on nerves. In other embodiments, the formulations and methods are used to treat low back pain, cancer pain, osteoarthritis pain, fibromyalgia pain and postoperative pain. In various other embodiments, the formulations and methods are used to treat pain associated with inflammation, bone pain, and joint disease. The formulations and methods of the invention may be used to treat pain caused by a variety of conditions, including, but not limited to, pain after surgery or trauma, pain associated with a medical illness and the like.

The present invention encompasses formulations that can be administered to provide two opioids. An objective of the present invention is to activate certain opioid receptors in the brain by one opioid, and stage the arrival of a second opioid at some timepoint after that receptor is occupied by the first opioid. A dual-opioid extended-release tablet is designed to accomplish this. For example, in formulations that contain oxycodone and morphine, there is a need to delay the release of morphine until the oxycodone is at the receptor by at least one-half hour, and preferably more than one hour. There is also a need to re-supply oxycodone for uptake into the brain at roughly the same rate of elimination from the CNS compartment. It is anticipated that both the delay and the rate of release of oxycodone should approximate one another in the delayed, modified-release pellet components described herein as well as formulations that incorporate the pellets such as, but not limited to, tablets and capsules.

### Compounds Having Opioid Receptor Agonist Activity

The components of the pharmaceutical formulations may comprise a compound having opioid receptor agonist activity. Such compounds may have agonist activity toward the μ-, κ-, σ-, or δ-opioid receptors, including other classified receptor subtypes. The compounds having opioid receptor agonist activity may be naturally occurring, semi-synthetic or fully synthetic opiate compounds, derivatives or analogs thereof, or pharmaceutically acceptable salts, esters or prodrugs thereof. Naturally occurring opiates are alkaloid compounds that are found in the resin of the opium poppy, and include morphine, codeine and thebaine. Semi-synthetic or fully synthetic opiates include, but are not limited to, dihydromorphine, heterocodeine, dihydrocodeine, dihydromorphinone, dihydrocodeinone, 3,6-diacetyl morphine, morphinone, 6-desoxymorphine, heroin, oxymorphone, oxycodone, 6-methylene-dihydromorphine, hydrocodone, etorphine, bupemorphine, naloxone or naltrexone.

Compounds having μ-opioid receptor agonist activity may include, but are not limited to, morphine (and structurally related analogs and derivatives), alvimopan, buprenorphine, codeine, 6-desomorphine, dihydromorphine, dihydromorphinone, dihydrocodeine, dihydrocodeinone, 3,6-diacetylmorphine, 6-methylene-dihydromorphine, diphenoxylate, drotebanol, eseroline, etorphine, fentanyl, hydrocodone, levophenacylmorphan, methadone, oxymorphone, nicomorphine, pethidine, picenadol, tapentadole, thebaine, and trimebutane.

Compounds having κ-opioid receptor agonist activity may include, but are not limited to, asimadoline, butorphanol, bremazocine, cyclazocine, dextromethorphan, dynorphin, enadoline, ketazocine, nalbuphine, nalfurafine, norbuprenorphine, oxycodone, pentazocine, salvinorin A, 2-methoxymethyl salvinorin B and its ethoxymethyl and fluoroethoxymethyl homologues, spiradoline, and tifluadom.

Compounds having δ-opioid receptor agonist activity may include, but are not limited to, deltorphin, ethoxymetopon, leu-enkephalin, met-enkephalin, mitragyna speciosa (kratom), mitragynine, mitragynine-pseudoindoxyl, N-phenethyl-14-norbuprenorphine, norclozapine, and 7-spiroindanyloxymorphone.

In certain embodiments, the compound is selected from morphine, codeine, hydromorphone, hydrocodone, oxycodone, dihydrocodeine, dihydromorphine, oxymorphone, mixtures thereof, and pharmaceutically acceptable salts thereof.

Salts include, but are not limited to, hydrochloride, sulfate, bisulfate, tartrate, nitrate, citrate, bitratrate, phosphate, malate, maleate, hydrobromide, hydroiodide, fumarate, succinate and the like.

The components of the pharmaceutical formulations may contain more than one compound, such that the more than one compound is present in a ratio by weight. For example, the components may comprise two compounds, such that the compounds are present in a 2:1, 2:2, 2:3, 2:5, 3:1, or 3:4 weight ratio.

In particular embodiments, the compounds are morphine and oxycodone, or pharmaceutical salts thereof, in ratio of about 3:2 by weight. Pharmaceutical formulations comprising compounds that contain morphine and oxycodone, or pharmaceutical salts thereof, in ratio of about 3:2 by weight, can administer up to a total amount of 18 mg morphine and 12 mg oxycodone per dosage. In some embodiments, pharmaceutical formulations comprising compounds that contain morphine and oxycodone, or pharmaceutical salts thereof, in ratio of about 3:2 by weight, can administer up to an amount of about 600 mg morphine, or pharmaceutical salts thereof, and about 400 mg oxycodone, or pharmaceutical salts thereof, per day.

### Release Profiles and Characteristics of the Components

At least one of the components in the pharmaceutical formulations comprises a compound having opioid receptor agonist activity and has a controlled release profile.

The formulations may comprise additional components, wherein the additional components may have an immediate release profile or a controlled release profile for the compound.

The term “immediate release” as used herein refers to a release profile in which there is substantially no delay in the release of the compound for absorption.

The term “controlled release” as used herein refers to a release profile in which there is a modification in the release of the compound as compared to an immediate release profile. Types of controlled release profiles include delayed release, extended release, and pulsatile release profiles.

The term “delayed release” as used herein refers to a release profile in which there is a delay in the release of the compound for absorption.

The term “extended release” as used herein refers to a release profile in which the active compound is released at such a rate that blood levels are maintained within the therapeutic range, but below toxic levels, over a period of time of about 8 hours, or about 10 hours, or about 12 hours, or about 15 hours, or about 20 hours, or about 24 hours or about 30 hours, or about 35 hours, or even longer. The term “extended release” differentiates release profile in accordance with the invention from “immediate release” and “delayed release” release profiles. As used herein, “delayed-extended release” refers to release profiles in which release of the active compound is delayed, but is still extended greater than “immediate release” release profiles.

The term “pulsatile release” as used herein refers to a release profile in which the compound is released at intervals for absorption.

### Immediate Release Component

The immediate release component may provide about 1% to about 50% of the total dosage of the compound(s) to be delivered by the pharmaceutical formulation. For example, the immediate release component may provide at least about 5%, or about 10% to about 30%, or about 45% to about 50% of the total dosage of the compound(s) to be delivered by the formulation. In alternate embodiments, the immediate release component provides about 2, 4, 5, 10, 15, 20, 25, 30, 35, 40, 45 or 50% of the total dosage of the compound(s) to be delivered by the formulation.

The immediate release component may be a mixture of ingredients that breaks down quickly after administration to release the opioid compound. This can take the form of, for example, granules, particles, powders, liquids and pellets.

### Controlled Release Component

The controlled release component may provide about 30-95% of the total dosage of the compound(s) to be delivered by the pharmaceutical formulation. For example, the immediate release component may provide about 70-90%, or about 80% of the total dosage of the compound(s) to be delivered by the pharmaceutical formulation. In alternate embodiments, the controlled release component provides about 30, 35, 40, 45, 50, 55, 60, 65, 70, 75, 80, 85, 90 or 95% of the total dosage of the compound(s) to be delivered by the formulation.

A controlled release component may have a Tₘₐₓ of about 1 to about 25 hours following repeated or single administration, or about 20, 17, 15, 12, 11, 8, 6, 5, 4, 3, 2 or 1 hours following administration.

In certain embodiments, a controlled release component may have a Tₘₐₓ of about 4.5 to about 8 hours after repeated administration, or about 5 to about 6 hours after repeated administration, or about 6 hours after repeated administration.

A controlled release component may have may have a Tₘᵢₙ about 10 to about 25 hours after repeated administration, or about 12, 13, 14, 15, 16, 17, 18, 19 or 20 hours following administration.

In certain embodiments, a controlled release component may have a Tₘᵢₙ of about 13 to about 16 hours after repeated administration, or about 14 hours after repeated administration.

Dissolution of a controlled release component release about 0 to about 20% of the compound or salt thereof after two hours, or releases about 15 to about 60% of the compound or salt thereof after four hours, or releases about 25 to about 80% of the compound or salt thereof after six hours, or releases about 35 to about 85% of the compound or salt thereof after eight hours, or releases about 45 to about 95% of the compound or salt thereof after ten hours, or releases about 60 to about 100% of the compound or salt thereof after twelve hours, as measured in a USP type I apparatus at 37° C. in water at 50 rpm.

A controlled release component may comprise about 2 mg to about 80 mg of the compound. When controlled release component comprises about 2 mg, the controlled release component may provide a mean Cₘₐₓ of about 1 to about 3 ng/mL, or about 2 ng/mL, after repeated administration. The AUC₂₄ may be about 14.7 ng·hr/mL to about 23.0 ng·hr/mL, or about 15.8 ng·hr/mL to about 21.0 ng·hr/mL, or about 17.1 ng·hr/mL to about 19.7 ng·hr/mL, after single administration.

When a controlled release component comprises about 5 mg, the controlled release component may provide a mean Cₘₐₓ of about 3 to about 7 ng/mL, or about 5 ng/mL, after repeated administration. The AUC₂₄ may be about 40.2 ng·hr/mL to about 62.8 ng·hr/mL, or about 43.2 ng·hr/mL to about 57.2 ng·hr/mL, or about 46.7 ng·hr/mL to about 53.7 ng·hr/mL, after single administration.

When a controlled release component comprises about 10 mg, the controlled release component may provide a mean Cₘₐₓ of about 5 to about 15 ng/mL, or about 10 ng/mL, after repeated administration. The AUC₂₄ may be about 80.5 ng·hr/mL to about 125.9 ng·hr/mL, or about 86.6 ng·hr/mL to about 114.8 ng·hr/mL, or about 93.7 ng·hr/mL to about 107.7 ng·hr/mL, after single administration.

When a controlled release component comprises about 20 mg, the controlled release component may provide a mean Cₘₐₓ of about 10 to about 30 ng/mL, or about 20 ng/mL, after repeated administration. The AUC₂₄ may be about 166.0 ng·hr/mL to about 259.3 ng·hr/mL, or about 178.5 ng·hr/mL to about 236.6 ng·hr/mL, or about 193.0 ng·hr/mL to about 222.0 ng·hr/mL, after single administration.

When a controlled release component comprises about 40 mg, the controlled release component may provide a mean Cₘₐₓ of about 25 to about 55 ng/mL, or about 40 ng/mL, after repeated administration. The AUC₂₄ may be about 338.5 ng·hr/mL to about 528.9 ng·hr/mL, or about 363.9 ng·hr/mL to about 482.3 ng·hr/mL, or about 393.5 ng·hr/mL to about 452.7 ng·hr/mL, after single administration.

When a controlled release component comprises about 80 mg, the controlled release component may provide a mean Cₘₐₓ of about 50 to about 110 ng/mL, or about 80 ng/mL, after repeated administration. The AUC₂₄ may be about 868.4 ng·hr/mL to about 1356.9 ng·hr/mL, or about 933.5 ng·hr/mL to about 1237.5 ng·hr/mL, or about 1009.5 ng·hr/mL to about 1161.5 ng·hr/mL, after single administration.

In some embodiments, a controlled release component provides a mean Cₘᵢₙ of about 0.5 to about 40 ng/mL, or about 4 to about 15 ng/mL, after repeated administration.

In certain embodiments, Tₘₐₓ, Tₘᵢₙ, mean Cₘₐₓ, and Cₘᵢₙ may be determined after repeated administrations through steady state conditions. As used herein, the term “steady state” means that a plasma level for a given drug has been achieved and which is maintained with subsequent doses of the drug at a level which is at or above the minimum effective therapeutic level and is below the minimum toxic plasma level for compound. For opioid analgesics such as oxycodone, the minimum effective therapeutic level will be partially determined by the amount of pain relief achieved in a given patient. It will be well understood by those skilled in the medical art that pain measurement is highly subjective and great individual variations may occur among patients. It is clear that after the administration of each dose the concentration passes through a maximum and then again drops to a minimum.

The steady state may be described as follows: at the time t=0, the time the first dose is administered, the concentration C is also 0. The concentration then passes through a first maximum and then drops to a first minimum. Before the concentration drops to 0, another dose is administered, so that the second increase in concentration does not start at 0. Building on this first concentration minimum, the curve passes through a second maximum after the second dose has been administered, which is above the first maximum, and drops to a second minimum, which is above the first minimum. Thus, the blood plasma curve escalates due to the repeated doses and the associated step-by-step accumulation of active agent, until it levels off to a point where absorption and elimination are in balance. This state, at which absorption and elimination are in equilibrium and the concentration oscillates constantly between a defined minimum and a defined maximum, is called steady state.

### Active Agents of the Components

The one or more additional components may comprise one or more active agents. For example, the active agents may be any of the compounds having opioid receptor agonist activity as discussed herein.

The active agents may also comprise one or more non-opioid analgesic compound(s), or a mixture of one or more non-opioid analgesic compound(s) and one or more compound(s) with opioid receptor agonist activity, or pharmaceutically acceptable salts, esters or prodrugs thereof. Non-opioid analgesic compounds may act to alleviate pain by other mechanisms not associated with binding to an opioid receptor. For example, the non-opioid analgesic compound may be a non-steroidal anti-inflammatory compound (NSAID), examples of which can include, but are not limited to, piroxicam, lomoxicam, tenoxicam, salicylic acid (aspirin) and other salicylates such as diflunisal; 2-arylpropionic acids such as ibuprofen, carprofen, fenbufen, fenoprofen, flubiprofen, ketoprofen, ketorolac, loxoprofen, naproxen, oxaprozin, tiaprofenic acid and suprofen; n-arylanthranilic acids such as metenamic acid and meclofenamic acid; arylalkanoic acids such as diclofenac, aceclofenac, acemetacin, etodolac, idomethacin, sulindac and tolmetin and the like; or mixtures thereof.

The non-opioid analgesic compound may also be a COX-1 or COX-2 inhibitor compound including, but not limited to, celecoxib (Celebrex), etoricoxib, lumiracoxib, parecoxib, rofecoxib, valdecoxib, or mixtures thereof. The non-opioid analgesic may also be a calcium channel binding agent such as gabapentin or pregabalin, or a derivative, analog or prodrug thereof, or mixtures thereof.

In certain embodiments, the non-analgesic compound is gabapentin enacarbil (Solzira™), which is a prodrug of gabapentin with the chemical name 1-[[[[1-(2-Methyl-1-oxopropoxy)ethoxy]carbonyl]amino]methyl]cyclohexaneacetic acid. The structures of gabapentin, pregabalin and gabapentin enacarbil are shown below:

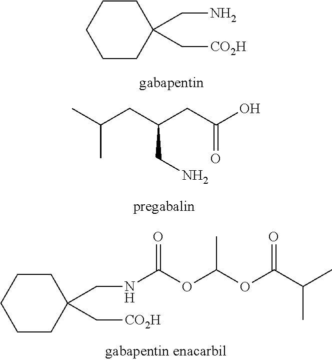

The active agents may further be one or more hybrid opioid compound(s), or a mixture of one or more hybrid opioid compound(s) and one or more compound(s) with opioid receptor agonist activity, or pharmaceutically acceptable salts, esters or prodrugs thereof. Hybrid opioid compounds are compounds formed by covalently binding together two or more opioid compounds with a linker component. The linker component may be stable or may hydrolyze under physiological conditions to provide the parent opioid compounds. Hybrid opioid compounds are described in U.S. Provisional Application Ser. No. 61/153,537 to Holaday et al., filed Feb. 18, 2009. Hybrid opioid compounds are also described in International Patent Application Publication No. WO 2006/073396 to Portoghese et al.

The hybrid opioid compound may comprise two or more compounds having opioid receptor agonist activity, linked by a covalent linker component. The hybrid opioid compound may also comprise a compound having opioid receptor agonist activity linked to a non-opioid active agent including, but not limited to, a non-opioid analgesic compound as described above. In some embodiments, the non-opioid active agent is gabapentin, pregabalin, or gabapentin enacarbil.

The hybrid opioid compound may comprise two or more opiate compounds bonded together by a covalent linker. The opiate compounds may include, but are not limited to, the opiate compounds described above.

The active compounds may be bonded to the linker components by various chemical bonds, preferably at a position on the active agent that does not impair the biological activity of the active agent. Typically, the active agents may be bonded to the linker by a reactive group on the active compound or at a position that may be activated to react with a linker component.

### Preparing the Components

To obtain the components of the pharmaceutical compositions described herein, a combination of excipients is used at appropriate concentrations to provide properties and desired pharmacokinetics. Excipients used in the pharmaceutical compositions described herein are commercially-available, and listed in either the USP or NF. Excipients are selected that will contribute to the function and purpose of each of the active intermediate components and also to the final component. One of ordinary skill will appreciate that the concentrations of these excipients used may be increased or decreased as desired to increase or decrease specific properties in a final opioid formulation. Coating materials used herein are also commercially-available and listed in the USP or NF which are incorporated herein by reference.

The technology used to produce a compound-opioid extended-release tablet described herein is a combination of known pharmaceutical manufacturing processes. The unit processes for the manufacture of each active intermediate have been used in several commercially-available products, and therefore are scalable. Two important aspects in producing the compound-opioid extended-release tablet are in the manufacture and performance of the different types of delayed, modified-release pellets. In the example of a dual opioid oxycodone/morphine compound product, the manufacture and performance of the delayed, modified-release oxycodone pellets and the delayed, modified-release morphine pellets is similarly important. These pellets should perform the same as free-flowing, untableted pellets as after tablet compaction. This important feature is best accomplished by adequately plasticizing the coating network to avoid cracking and brittle fracture of the coatings when under compression during tablet compaction.

The materials to be added to the compound(s) for the immediate release component can be, but are not limited to, microcrystalline cellulose, corn starch, pregelatinized starch, potato starch, rice starch, sodium carboxymethyl starch, hydroxypropylcellulose, hydroxypropylmethylcellulose, hydroxyethylcellulose, ethyl-cellulose, chitosan, hydroxychitosan, hydroxymethylated chitosan, cross-linked chitosan, cross-linked hydroxymethyl chitosan, maltodextrin, mannitol, sorbitol, dextrose, maltose, fructose, glucose, levulose, sucrose, polyvinylpyrrolidone (PVP), acrylic acid derivatives (Carbopol, Eudragit, etc.), polyethylene glycols, such a low molecular weight PEGs (PEG2000-10000) and high molecular weight PEGs (Polyox) with molecular weights above 20,000 daltons. It may be useful to have these materials present in the range of 1.0 to 60% (W/W).

In addition, it may be useful to have other ingredients in this system to aid in the dissolution of the drug, or the breakdown of the component after ingestion or administration. These ingredients can be surfactants, such as sodium lauryl sulfate, sodium monoglycerate, sorbitan monooleate, sorbitan monooleate, polyoxyethylene sorbitan monooleate, glyceryl monostearate, glyceryl monooleate, glyceryl monobutyrate, one of the non-ionic surfactants such as the Pluronic line of surfactants, or any other material with surface active properties, or any combination of the above. These materials may be present in the rate of 0.05-15% (W/W).

The materials in controlled release components are the same as the materials in the immediate release component, but with additional polymers integrated into the component, or as coatings over the pellet or granule. The kind of materials useful for this purpose can be, but are not limited to, ethylcellulose, hydroxypropylmethylcellulose, hydroxypropylcellulose, hydroxyethylcellulose, carboxymethylcellulose, methylcellulose, nitrocellulose, Eudragit R, and Eudragit RL, Carbopol, or polyethylene glycols with molecular weights in excess of 8,000 daltons. These materials can be present in concentrations from 4-20% (W/W).

In certain embodiments, components may have pH-sensitive delayed release profiles or non-pH sensitive delayed release profiles. Materials in the pH-sensitive delayed release components may be the same as the materials in the immediate release component, but with additional polymers integrated into the component, or as coatings over the pellet or granule. The kind of materials useful for this purpose can be, but are not limited to, cellulose acetate pthalate, Eudragit L, and other pthalate salts of cellulose derivatives. These materials can be present in concentrations from 4-20% (W/W).

Materials in the pH-sensitive delayed release components may be the same as the materials in the immediate release component, but with additional polymers integrated into the component, or as coatings over the pellet or granule. The kind of materials useful for this purpose can be, but are not limited to, polyethylene glycol (PEG) with molecular weight above 4,000 daltons (Carbowax, Polyox), waxes such as white wax or bees wax, paraffin, acrylic acid derivatives (Eudragit), propylene glycol, and ethylcellulose. Typically these materials can be present in the range of 0.5-25% (W/W) of this component.

### Pharmaceutical Formulations

The pharmaceutical formulations may comprise one or more components having one or more release profiles. Each of the components may comprise the same compound(s), may comprise different compound(s), or a mixture thereof (e.g., some components have the same compounds, other components have different compounds, within the same formulation). In addition, components may comprise active agents as described herein.

For example, the formulations may comprise at least one component, such that the one component has a controlled release profile.

The formulations may also comprise at least two components (a first and second component), such that each components has a different release profile. For example, the second of the at least two components initiates release of the compound(s) contained therein at least one hour after the first component, with the initiation of the release therefrom generally occurring no more than six hours after initiation of release of compound(s) from the first component.

The formulations may also comprise at least three components (a first, second, and third component). The first component may be an immediate release component whereby initiation of release of the compound(s) therefrom is not substantially delayed after administration of the formulation. The second and third components are controlled release components, whereby the release of the compound(s) may be delayed. The controlled release components may be a pH sensitive or a non-pH sensitive delayed component, depending on the type of formulation. The compound(s) released from the delayed release components may be delayed until after initiation of release of the compound(s) from the immediate release component. For example, the compound(s) release from the second component may achieve a Cₘₐₓ at a time after the compound(s) released from the immediate release component may achieve a Cₘₐₓ in the serum. The compound(s) released from the third component may achieve a Cₘₐₓ in the serum after the Cₘₐₓ of the compound(s) released from the second component.

In certain embodiments, the immediate release component may produce a Cₘₐₓ for the compound(s) released therefrom within from about 0.5 to about 2 hours, with the second component producing a Cₘₐₓ for the compound(s) released therefrom in no more than about four hours. In general, the Cₘₐₓ for such a second component may be achieved no earlier than two hours after administration of the formulation; however, it is possible to achieve Cₘₐₓ in a shorter period of time by adjusting the concentration of excipients and/or coatings described herein to achieve a formulation with a desired pharmacokinetic profile.

In certain embodiments, release of compound(s) from the third component may be started after initiation of release of compound(s) from both the first and second components. In some embodiments, Cₘₐₓ for compound(s) released from the third component may be achieved within eight hours.

The formulations may also comprise at least four components (a first, second, third, and fourth component), with each of the at least four components having different release profiles. For example, the compound(s) released from each of the at least four different components may achieve a Cₘₐₓ at a different time.

The formulations may also comprise at least five components (a first, second, third, fourth, and fifth component). The first component may be an immediate release component of a first compound or a first set of compounds, while the second and third components may be controlled release components of the first compound or a first set of compounds. The fourth and fifth components may be controlled release components of a second compound or a second set of compounds. As an example, in certain embodiments, the first compound may be oxycodone and the second compound may be morphine.

In certain embodiments, the formulation may be in the form of a capsule, comprising components that are in the form of separate tablets or pellets. Thus, for example, an immediate release component may be in the form of a tablet or pellet, and controlled release components may be in the form of other tablets or pellets, each of which provides for a delayed release of the compound(s) contained therein, whereby the Cₘₐₓ of the compound(s) released from each of the pellets, or tablets containing the pellets, is reached at different times, with the Cₘₐₓ of the formulation being achieved in less than twelve hours.

In certain embodiments, the pharmaceutical formulation itself will comprise a controlled release profile. For example, Cₘₐₓ for all of the compound(s) released from the formulation may be achieved in about 20, 17, 15, 12, 11, 8, 6, 5, 4, 3, 2 or 1 hours following administration of the formulation. In some embodiments, Cₘₐₓ may be achieved in less than 2, 1 or 0.5 hours following administration of the formulations. In other embodiments, Cₘₐₓ may be achieved in greater than 4.5, 5, 6, 7, 8, 9, or 10 hours following administration of the component.

The formulation may have a Tₘₐₓ of about 1 to about 25 hours following repeated or single administration, or about 20, 17, 15, 12, 11, 8, 6, 5, 4, 3, 2 or 1 hours following administration.

In certain embodiments, Tₘₐₓ may be about 4.5 to about 8 hours, or about 5 to about 6 hours, or about 6 hours, after repeated administration. In some embodiments, the repeated administration is under steady state conditions.

The formulation may comprise about 1 mg to about 100 mg of the compounds(s), or may comprise about 2 mg to about 80 mg of the compound(s). When the formulation comprises about 2 mg, the controlled release component may provide a mean Cₘₐₓ of about 1 to about 3 ng/mL, or about 2 ng/mL, after repeated administration. The AUC₂₄ may be about 14.7 ng·hr/mL to about 23.0 ng·hr/mL, or about 15.8 ng·hr/mL to about 21.0 ng·hr/mL, or about 17.1 ng·hr/mL to about 19.7 ng·hr/mL, after single administration.

When the formulation comprises about 5 mg, the controlled release component may provide a mean Cₘₐₓ of about 3 to about 7 ng/mL, or about 5 ng/mL, after repeated administration. The AUC₂₄ may be about 40.2 ng·hr/mL to about 62.8 ng·hr/mL, or about 43.2 ng·hr/mL to about 57.2 ng·hr/mL, or about 46.7 ng·hr/mL to about 53.7 ng·hr/mL, after single administration.

When the formulation comprises about 10 mg, the controlled release component may provide a mean Cₘₐₓ of about 5 to about 15 ng/mL, or about 10 ng/mL, after repeated administration. The AUC₂₄ may be about 80.5 ng·hr/mL to about 125.9 ng·hr/mL, or about 86.6 ng·hr/mL to about 114.8 ng·hr/mL, or about 93.7 ng·hr/mL to about 107.7 ng·hr/mL, after single administration.

When the formulation comprises about 20 mg, the controlled release component may provide a mean Cₘₐₓ of about 10 to about 30 ng/mL, or about 20 ng/mL, after repeated administration. The AUC₂₄ may be about 166.0 ng·hr/mL to about 259.3 ng·hr/mL, or about 178.5 ng·hr/mL to about 236.6 ng·r/mL, or about 193.0 ng·hr/mL to about 222.0 ng·hr/mL, after single administration.

When the formulation comprises about 40 mg, the controlled release component may provide a mean Cₘₐₓ of about 25 to about 55 ng/mL, or about 40 ng/mL, after repeated administration. The AUC₂₄ may be about 338.5 ng·hr/mL to about 528.9 ng·hr/mL, or about 363.9 ng·hr/mL to about 482.3 ng·hr/mL, or about 393.5 ng·hr/mL to about 452.7 ng·hr/mL, after single administration.

When the formulation comprises about 80 mg, the controlled release component may provide a mean Cₘₐₓ of about 50 to about 110 ng/mL, or about 80 ng/mL, after repeated administration. The AUC₂₄ may be about 868.4 ng·hr/mL to about 1356.9 ng·hr/mL, or about 933.5 ng·hr/mL to about 1237.5 ng·hr/mL, or about 1009.5 ng·hr/mL to about 1161.5 ng·hr/mL, after single administration.

In certain embodiments, Cₘᵢₙ may occur within about 12 to about 18 hours following administration of the component during steady-state conditions. In some embodiments, Cₘᵢₙ may occur at about 12, 13, 14, 15, 16, 17, 18, 19 or 20 hours following administration of the formulation. In some embodiments, Cₘᵢₙ may occur less than about 10, 9, 8, 7, 6, 5, or 4 hours following administration of the formulation. In some embodiments, Cₘᵢₙ may occur at greater than about 14, 15, 16, 17, 18, 19, or 20 hours following administration of the formulation. In particular embodiments, the Cₘᵢₙ that occurs more than about 12 hours after administration, may occur up to about 1, 2, 3, or 4 hours after the administration of a formulation that has not yet been absorbed into the bloodstream.

The certain embodiments, the formulation may have a Tₘᵢₙ about 10 to about 25 hours after repeated administration, or about 12, 13, 14, 15, 16, 17, 18, 19 or 20 hours following administration.

In certain embodiments, the formulation may have a Tₘᵢₙ of about 13 to about 16 hours after repeated administration, or about 14 hours after repeated administration.

In some embodiments, the formulation may provide a mean Cₘᵢₙ of about 0.5 to about 40 ng/mL, or about 4 to about 15 ng/mL, after repeated administration.

Dissolution of the formulation releases about 0 to about 20% of the compound(s) or salt thereof after two hours, or releases about 15 to about 60% of the compound(s) or salt thereof after four hours, or releases about 25 to about 80% of the compound(s) or salt thereof after six hours, or releases about 35 to about 85% of the compound(s) or salt thereof after eight hours, or releases about 45 to about 95% of the compound(s) or salt thereof after ten hours, or releases about 60 to about 100% of the compound(s) or salt thereof after twelve hours, as measured in a USP type I apparatus at 37° C. in water at 50 rpm.

It is to be understood that when it is disclosed herein that a formulation initiates release after another component, such terminology means that the formulation is designed and is intended to produce such later initiated release. It is known in the art, however, notwithstanding such design and intent, some “leakage” of compound(s) may occur. Such “leakage” is not “release” as used herein.

In particular embodiments, the pharmaceutical formulation may comprise one or more components that contain two opioid compounds in a 2:1, 2:2, 2:3, 2:5, 3:1, or 3:4 weight ratio. In certain embodiments, the components may comprise morphine and oxycodone in about a 3:2 weight ratio.

As an example, the pharmaceutical formulation may comprise a controlled release component comprising a mixture of morphine and oxycodone, and an immediate release component comprising oxycodone. In some embodiments, the Tₘₐₓ of oxycodone in the immediate release component may be from about 10 minutes to about one hour after ingestion. In other embodiments, the Tₘₐₓ will be from about 10 minutes to about 30 minutes or 45 minutes. The controlled release component may be released at a slower rate and over a longer period of time. For example, in some embodiments, the controlled release component may release effective amounts of the mixture of morphine and oxycodone over 12 hours. In other embodiments, the controlled release component may release effective amounts of morphine and oxycodone over 4 hours or over 8 hours. In still other embodiments, the controlled release component t may release effective amounts of morphine and oxycodone over 15, 18, 24 or 30 hours.

In some embodiments, the later released active agents may be released from the pharmaceutical formulation in pulses so that pulses of the compounds are released at intervals after ingestion of the formulation. For example, in certain embodiments, controlled release component may release a first pulse of the later released active agents about 0.5-1 hour after ingestion, followed by a second pulse after about of 4 hours after ingestion and a third pulse of drug after about 8 hours after ingestion.

### Preparing Formulations

In one aspect, the pharmaceutical compositions are tablets and capsules for oral administration. These tablets or capsules may contain conventional excipients such as binding agents, fillers, lubricants, disintegrants, or wetting agents. In one aspect the tablets or capsules are coated according to methods well known in the art.

The granulation that will best serve this purpose will be highly deformable during compaction, thereby minimizing as much as possible any leakage from the coated pellets before the designated time of release. In one embodiment, it may be desirable to have a brief lag, or delay in the initial burst, or release of oxycodone in the immediate release bolus portion of the formulation. In some embodiments, the tablet is less than about 500 mg, about 450 mg, about 400 mg, about 350 mg, about 300 mg, about 250 mg, about 200 mg, about 150 mg, about 100 mg, about 50 mg, about 25 mg, or about 10 mg weight, and the drug load is about 20%, about 15%, about 10%, about 5% (w/w) or less of the formulation. In one embodiment, the goal would be to have as efficient a tablet size as possible, while affording good uniformity and integrity of the pellets in the tablet.

The disintegrant used in the tablet of the present invention is not particularly limited, as far as it is a disintegrant used for pharmaceutical preparations. Examples can include crospovidone, crystalline cellulose, hydroxypropylcellulose with a low degree of substitution, croscarmellose sodium, carmellose calcium, carboxystarch sodium, carboxymethyl starch sodium, potato starch, wheat starch, corn starch, rice starch, partly pregelatinized starch, and hydroxypropyl starch. One or two or more of these can be used. Crospovidone is particularly preferable. The sort of disintegrant used for coating granules according to the present invention may be identical to or different from that used inside the granules.

Examples of pharmaceutically acceptable additives used in the tablet of the present invention can include excipients, lubricants, pH adjusters, taste-masking agents, sweeteners, acidifiers, refrigerants, foaming agents, preservatives, fluidizers, antioxidants, colorants, stabilizers, surfactants, buffering agents, flavors, binders and drug solubilizers. A person skilled in the art may immediately list specific examples of these additives.

These additives can be appropriately formulated in the inside of a granule, in the outside of a granule coated with a disintegrant, in the coating of a disintegrant and in all these, as far as they do not damage the advantages of the present invention.

Any lubricant used for pharmaceutical preparation can be used without limitation. Examples of the lubricant used in the tablet of the present invention can include light anhydrous silicic acid, magnesium stearate, stearic acid, calcium stearate, aluminum stearate, aluminum monostearate, sucrose fatty acid esters, polyethylene glycol, sodium stearyl fumarate, stearyl alcohol, talc, titanium oxide, hydrous silicon dioxide, magnesium silicate, synthetic aluminum silicate, calcium hydrogen phosphate, hardened castor oil, hardened rapeseed oil, Carnauba Wax, bees wax, microcrystalline wax and sodium lauryl sulfate. One or two or more kinds of these lubricants can be used. Among these, it is preferable to use one or more selected from light anhydrous silicic acid and magnesium stearate. Particularly, a combination of silicic anhydride contained in the inside of a granule and magnesium stearate contained in the outside of the granule is preferable.

When the formulations are in the form of a tablet, the shape of the tablet is not particularly limited, as far as it can be produced without difficulty using an ordinary manufacturing apparatus or a manufacturing apparatus with some modifications. A disc shape that is a general concept for tablets can be mentioned as a typical example. The whole size is not particularly limited. For example, the shorter diameter (diameter for a disc tablet) is appropriately in the range of 6 to 20 mm, preferably 8 to 12 mm The thickness is neither particularly limited, but appropriately 1 to 10 mm, preferably 2 to 8 mm.

In some embodiments, it may be desirable to have the initial short delay accomplished by adding a delayed-release coating to the tablet which would also serve as a taste-masking agent. This coating may be white, or colored or clear or opaque if desired. An identifying NDC code (in the United States) or similar identifying code may also be printed on the tablet if desired.

The compound used in the tablet of the present invention may be coated with a filmcoating agent, an excipient, a binder, a lubricant, or the like depending on its properties and a plasticizer may be added.

### Anti-Abuse Properties

In another aspect of the invention, the pharmaceutical compositions described herein possess properties that are useful in deterring their use to create compositions that are likely to be used for nonmedical purposes, or as a drug of abuse.

Intentional or inadvertent tampering from extended release formulations will rapidly deliver a massive dose (as a result of converting the sustained release product into an immediate release form) and produce profound a variety of serious and life threatening side effects, including respiratory depression and failure, sedation, cardiovascular collapse, coma and death.

Addicts and recreational drug users commonly use extended release opioids by a variety of routes of administration. Commonly used methods include (a) parenteral (e.g., intravenous injection), (b) intranasal (e.g., snorting), and (c) episodic or repeated oral ingestion of intact or crushed tablets or capsules.

One mode of abuse involves the extraction of the opioid from the component by first mixing the table or capsule with a suitable solvent (e.g., water or alcohol), and then filtering and/or extracting the opioid component from the mixture for intravenous injection. Another mode of abuse of extended release opioids involves dissolving the drug in water, alcohol or another “recreational solvent” to hasten its release and to ingest the contents orally, in order to provide high peak concentrations and maximum euphoriant effects.

The term “tampering” means any manipulation by mechanical, thermal and/or chemical means which changes the physical properties of the component, e.g., to liberate the opioid for immediate release if it is in sustained release form, or to make the opioid agonist available for inappropriate use such as administration by an alternate route, e.g., parenterally. The tampering can be, e.g., by means of crushing, shearing, grinding, mechanical extraction, solvent extraction, solvent immersion, combustion, heating or any combination thereof.

The term “abuse,” “opioid agonist abuse” or “opioid abuse” in the context of the present invention, when it refers to the effects of opioid agonists in causing such, includes intermittent use, recreational use and chronic use of opioid agonists alone or in conjunction with other drugs: (i) in quantities or by methods and routes of administration that do not conform to standard medical practice; (ii) outside the scope of specific instructions for use provided by a qualified medical professional; (iii) outside the supervision of a qualified medical professional; (iv) outside the approved instructions on proper use provided by the drug's legal manufacturer; (v) which is not in specifically approved components for medical use as pharmaceutical agents; (vi) where there is an intense desire for and efforts to procure same; (vii) with evidence of compulsive use; (viii) through acquisition by manipulation of the medical system, including falsification of medical history, symptom intensity, disease severity, patient identity, doctor shopping, prescription forgeries; (ix) where there is impaired control over use; (x) despite harm; (xi) by procurement from non-medical sources; (xii) by others through sale or diversion by the individual into the non-medical supply chain; (xiii) for medically unapproved or unintended mood altering purposes.

The term “abuse resistant,” “abuse deterrent” and “deter abuse” are used interchangeably in the context of the present invention and include pharmaceutical compositions and methods that (i) resist, deter, discourage, diminish, delay and/or frustrate the intentional, unintentional or accidental physical manipulation or tampering of the component (e.g., crushing, shearing, grinding, chewing, dissolving, melting, needle aspiration, inhalation, insufflation, extraction by mechanical, thermal and chemical means, and/or filtration); (ii) resist, deter, discourage, diminish, delay and/or frustrate the intentional, unintentional or accidental use or misuse of the component outside the scope of specific instructions for use provided by a qualified medical professional, outside the supervision of a qualified medical professional and outside the approved instructions on proper use provided by the drug's legal manufacturer (e.g., intravenous use, intranasal use, inhalational use and oral ingestion to provide high peak concentrations); (iii) resist, deter, discourage, diminish, delay and/or frustrate the intentional, unintentional or accidental conversion of an extended release component of the invention into a more immediate release form; (iv) resist, deter, discourage, diminish, delay and/or frustrate the intentional and iatrogenic increase in physical and psychic effects sought by recreational drug users, addicts, and patients with pain who have an addiction disorder; (v) resist, deter, discourage, diminish, delay and/or frustrate the attempts at surreptitious administration of the component to a third party (e.g., in a beverage); (vi) resist, deter, discourage, diminish, delay and/or frustrate attempts to procure the component by manipulation of the medical system and from non-medical sources; (vii) resist, deter, discourage, diminish, delay and/or frustrate the sale or diversion of the component into the non-medical supply chain and for medically unapproved or unintended mood altering purposes; (viii) resist, deter, discourage, diminish, delay and/or frustrate intentional, unintentional or accidental attempts at otherwise changing the physical, pharmaceutical, pharmacological and/or medical properties of the component from what was intended by the manufacturer.

When the component of the pharmaceutical formulation is tampered, the pharmaceutical formulation reduces the amount of opioid agonist released in immediate release form, which in turn reduces the euphoric, pleasurable, reinforcing, rewarding, mood altering and toxic effects of the opioid agonist of the component.

In specific embodiments, the use of certain excipients such as Povidone (Kollidon 30) or Polyoxyl 35 Castor Oil (Cremophor EL™) or Sodium Lauryl Sulfate create an unusable gelatinous mass if tampered with. The addition of aqueous or hydroalcoholic solvents would render the pulverized excipient and drug mixture to a gelatinous mass that would be problematic for easy extraction of the opioid. The Cremophor, in admixture with the methacrylic acid polymers and cellulosic polymers are examples of prime ingredients that cause this feature of the invention.

Other methods of creating abuse-resistant opioid compositions are provided in U.S. published patent application US 20090082466, the teachings of which are incorporated herein by reference in their entirety.

### Administration of the Formulation

An aspect of the present invention is a method for treating pain comprising administering a formulation as described herein.

The formulations may be administered, for example, by any of the following routes of administration: sublingual, buccal, transmucosal, transdermal, parenteral, oral etc. In certain embodiments, the formulations may be prepared in a manner suitable for oral administration. Thus, for example, for oral administration, each of the components may be used as a pellet, granule, powder, liquid or a particle, which are then formed into a unitary pharmaceutical product, for example, in a capsule, or embedded in a tablet, or suspended in a liquid for oral administration. The term “formulation” as used herein also refers to a unitary pharmaceutical product containing at least one component.

In certain embodiments, the formulations are for oral administration and may be in the form of a tablet or a capsule or in the form of a multiple unit component. The formulations may be adapted for oral administration 1-6 times a day, normally 1-4 times daily such as 1-3 times, twice daily, or once daily. In the present context the term “once daily” is intended to mean that it is only necessary to administer the pharmaceutical composition once a day in order to obtain an effective therapeutic amount of the compound to provide a suitable therapeutic response.

The final dose of the compound(s) provided by administration of the formulation may be about, by weight, 100 mg, about 95 mg, about 90 mg, about 85 mg, about 80 mg, about 75 mg, about 70 mg, about 65 mg, about 60 mg, about 55 mg, about 50 mg, about 45 mg, about 40 mg, about 35 mg, about 30 mg, about 25 mg, about 20 mg, about 15 mg, about 12 mg, about 10 mg, about 8 mg, about 5 mg, about 4, mg, about 3 mg, about 2 mg, or about 1 mg.

The dosage of the opioid compound depends on the particular substance, the age, weight condition, etc., of the human or animal that will be treated with the composition, etc. All such factors are well known to a person skilled in the art.

### EXAMPLES

The present invention will be understood more readily by reference to the following examples, which are provided by way of illustration and are not intended to be limiting of the invention.

### Example 1

### Opioid Components

Components for use in pharmaceutical formulations were developed, as shown in Tables 1-8.

TABLE 1
Target Component 1 (oxycodone):

| | | | |
|---|---|---|---|
| Pellet Core: | | | |
| Oxycodone hydrochloride | USP | Drug Substance | 20% |
| Microcrystalline Cellulose | NF | Diluent | 75% |
| (Avicel ® PH-101) | | | |
| Povidone | USP | Binding Agent | 2-4% |
| (Kollidon 30) | | | |
| Polyoxyl 35 Castor Oil | NF | Wetting Agent | 0.5-1.5% |
| (Cremophor EL) | | | |
| Coating: | | | |
| Methacrylic Acid Copolymer | NF | Functional Film | 12% |
| Dispersion (Eudragit ® | | Sub-Coat | |
| L30D-55)¹ | | | |
| Hypromellose Acetate Succinate | NF | Functional Film | 48% |
| (AQOAT AS-HF) | | Over-Coat | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 2
Target Component 1 (morphine):

| | | | |
|---|---|---|---|
| Pellet Core: | | | |
| Morphine Sulfate | USP | Drug Substance | 20% |
| Microcrystalline Cellulose | NF | Diluent | 75% |
| (Avicel ® PH-101) | | | |
| Povidone | USP | Binding Agent | 2-4% |
| (Kollidon 30) | | | |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 3
Target Component 2 (oxycodone):

| | | | |
|---|---|---|---|
| Pellet Core: | | | |
| Oxycodone hydrochloride | USP | Drug Substance | 20% |
| Microcrystalline Cellulose | NF | Diluent | 70-75% |
| (Avicel ® PH-101) | | | |
| Methocel K50 | USP | Binding Agent | 4-9% |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 4
Target Component 2 (morphine):

| | | | |
|---|---|---|---|
| Pellet Core: | | | |
| Morphine Sulfate | USP | Drug Substance | 20% |
| Microcrystalline Cellulose | NF | Diluent | 70-75% |
| (Avicel ® PH-101) | | | |
| Methocel K50 | USP | Binding Agent | 4-9% |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 5
Target Component 3 (oxycodone):

| | | | |
|---|---|---|---|
| Pellet Core: | | | |
| Oxycodone hydrochloride | USP | Drug Substance | 20% |
| Microcrystalline Cellulose | NF | Diluent | 65-75% |
| (Avicel ® PH-101) | | | |
| Methocel E15 | USP | Binding Agent | 4-14% |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 6
Target Component 3 (morphine):

| | | | |
|---|---|---|---|
| Pellet Core: | | | |
| Morphine sulfate | USP | Drug Substance | 20% |
| Microcrystalline Cellulose | NF | Diluent | 65-75% |
| (Avicel ® PH-101) | | | |
| Methocel E15 | USP | Binding Agent | 4-14% |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 7
Target Component 4 (oxycodone):

| | | | |
|---|---|---|---|
| Pellet Core: | | | |
| Oxycodone hydrochloride | USP | Drug Substance | 20% |
| Microcrystalline Cellulose | NF | Diluent | 65-75% |
| (Avicel ® PH-101) | | | |
| Povidone | USP | Binding Agent | 4-14% |
| (Kollidon 25) | | | |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

TABLE 8
Target Component 4 (morphine):

| | | | |
|---|---|---|---|
| Pellet Core: | | | |
| Morphine sulfate | USP | Drug Substance | 20% |
| Microcrystalline Cellulose | NF | Diluent | 65-75% |
| (Avicel ® PH-101) | | | |
| Povidone | USP | Binding Agent | 4-14% |
| (Kollidon 25) | | | |
| Polyoxyl 35 Castor Oil | NF | Wetting Agent | 0.5-1.5% |
| (Cremophor EL) | | | |
| Coating: | | | |
| Methacrylic Acid | NF | Functional Film | 12% |
| Copolymer Dispersion | | Sub-Coat | |
| (Eudragit ® L30D-55)¹ | | | |
| Hypromellose Acetate | NF | Functional Film | 48% |
| Succinate (AQOAT | | Over-Coat | |
| AS-HF) | | | |
| Talc | USP | Antitacking Agent | 30% |
| Triethyl Citrate | NF | Plasticizer | 9.5% |
| Sodium Lauryl Sulfate | NF | Wetting Agent | 0.5% |
| Purified Water² | USP | Processing Agent | N/A |

¹Amount per tablet based on the solids content of the dispersion
²Removed during processing

### Example 2

### Pharmacokinetic Profile of Opioid Formulations

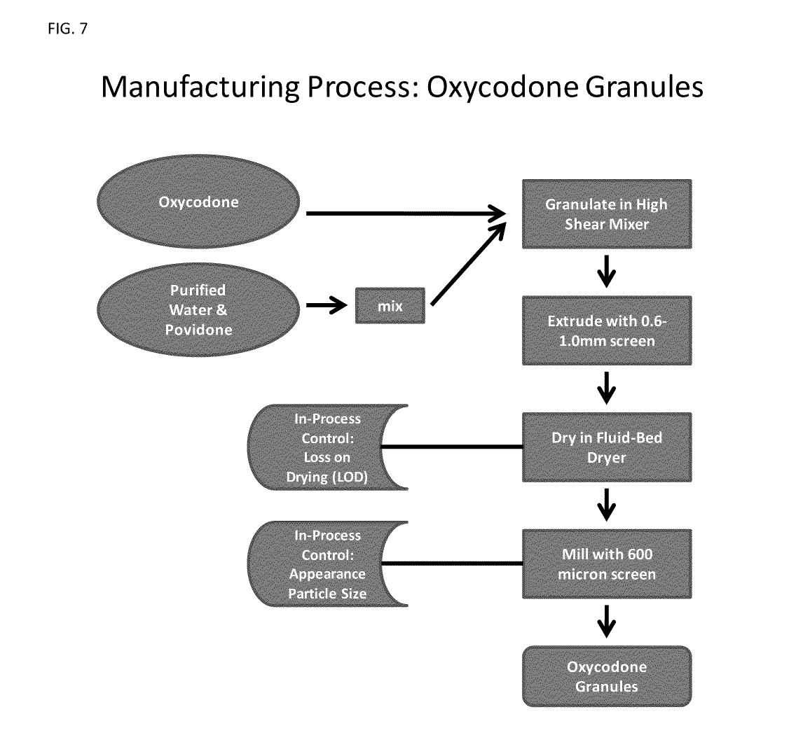

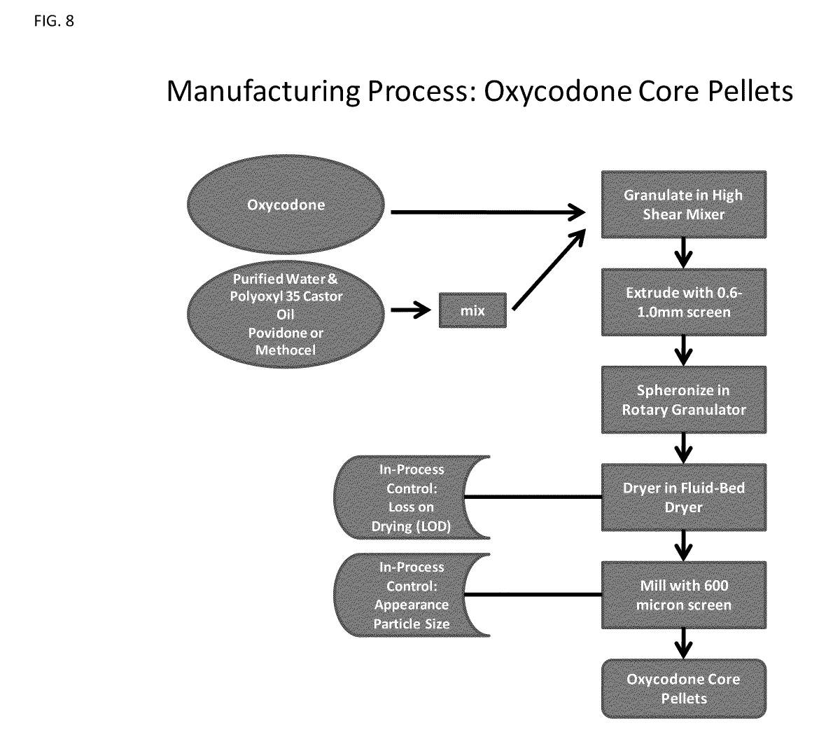

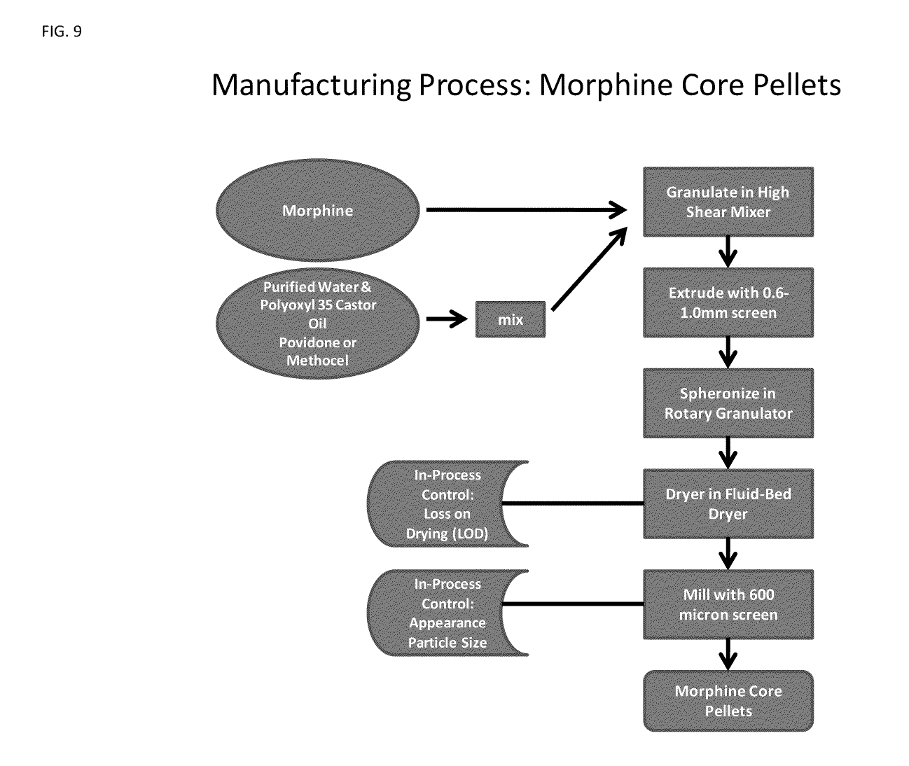

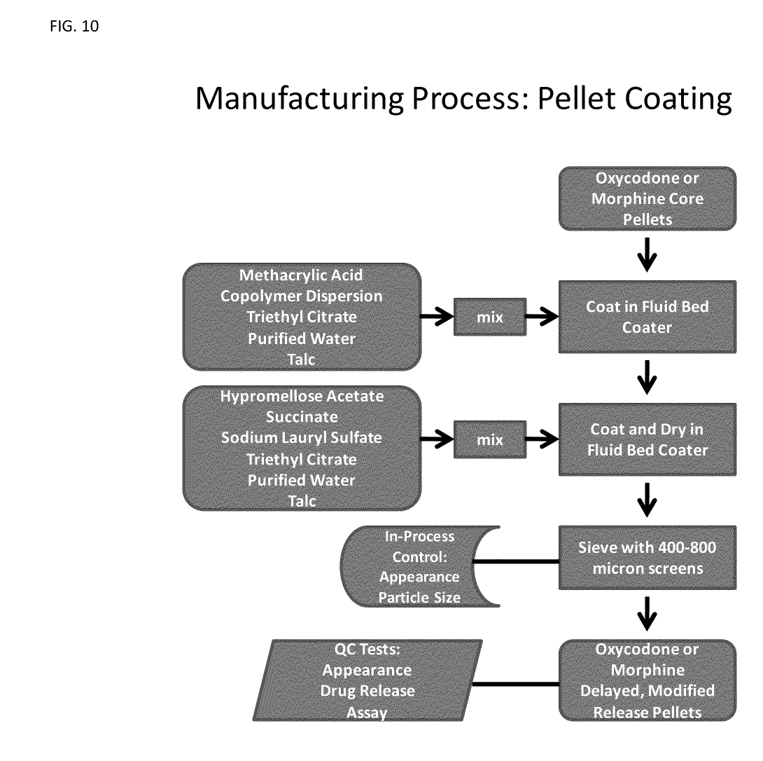

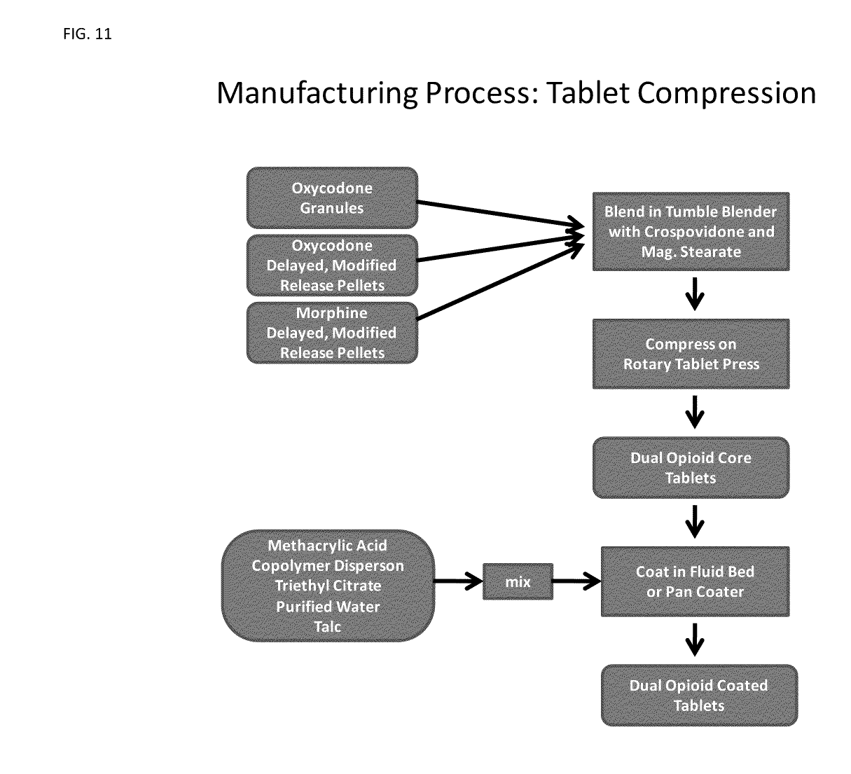

A. An oxycodone formulation is provided that has the following pharmacokinetic profile. The pharmacokinetic profile is achieved by adjusting the concentration of excipients using the methods described in the charts shown in FIGS. 7-11. This 8 mg oxycodone formulation has a Cₘₐₓ of 8 hours and a Cₘᵢₙ of 14 hours.

B. An oxycodone formulation is provided that has the following pharmacokinetic profile. The pharmacokinetic profile is achieved by adjusting the concentration of opioid compound and excipients using the methods described in the charts shown in FIGS. 7-11. This 8 mg oxycodone formulation has a Cₘₐₓ of 6 hours and a Cₘᵢₙ of 16 hours.

C. A dual opioid oxycodone/morphine formulation is provided that has the following pharmacokinetic profile. The pharmacokinetic profile is achieved by adjusting the concentration of opioid compound and excipients using the methods described in the charts shown in FIGS. 7-11. This 8 mg oxycodone/4 mg morphine formulation has a Cₘₐₓ of 6-20 hours for both opioids, and a Cₘᵢₙ of 15-26 hours for both opioids.

D. A dual opioid oxycodone/morphine formulation is provided that has the following pharmacokinetic profile. The pharmacokinetic profile is achieved by adjusting the concentration of opioid compound and excipients using the methods described in the charts shown in FIGS. 7-11. This 18 mg morphine/12 mg oxycodone formulation has a Cₘₐₓ of 6 hours, and a Cₘᵢₙ of 16 hours.

### Example 3

### Preparation of Extended Release Intermediate Pellets Formulations

Extended release intermediate pellet formulations A and B were prepared having the compositions as shown in Tables 9 and 10.

TABLE 9
Formulation A:

| | | | | |
|---|---|---|---|---|
| | Qual- | | | |
| Component | ity* | Function | mg/dose | % w/w |

Pellet Core

| | | | | |
|---|---|---|---|---|
| Oxycodone | USP | Drug Substance | 20.00 | 15.19 |
| hydrochloride | | | | |
| Microcrystalline | USP | Filter/Diluent | 75.00 | 56.96 |
| Cellulose | | | | |
| Povidone (Kollidon 30) | USP | Filter/Diluent | 4.00 | 3.04 |
| Polyoxyl 35 Castor Oil | NF | Lubricant | 1.00 | 0.76 |
| Purified Water | USP | Process Aid | — | — |

Pellet Barrier Film Coat

| | | | | |
|---|---|---|---|---|
| Ammonio Methacrylate | NF | Film Forming | 1.55 | 1.17 |
| Copolymer, Type A (RL) | | Agent | | |
| Ammonio Methacrylate | NF | Film Forming | 6.18 | 4.70 |
| Copolymer, Type B (RS) | | Agent | | |
| Triethyl Citrate | NF | Plasticizer | 0.77 | 0.59 |
| Magnesium Stearate | NF | Antitacking | 1.50 | 1.14 |
| | | Agent | | |
| Isopropyl Alcohol | USP | Process Aid | — | — |
| Purified Water | USP | Process Aid | — | — |

Pellet Enteric Film Coat

| | | | | |
|---|---|---|---|---|
| Methacrylic Acid | NF | Film Forming | 12.75 | 9.68 |
| Copolymer Disp., | | Agent | | |
| Type C | | | | |
| Triethyl Citrate | NF | Plasticizer | 1.28 | 0.97 |
| Talc | NF | Antitacking | 6.38 | 4.84 |
| | | Agent | | |
| Isopropyl Alcohol | USP | Process Aid | — | — |
| Purified Water | USP | Process Aid | — | — |

Dusting Powder

| | | | | |
|---|---|---|---|---|
| Colloidal Silicon | NF | Lubricant | 1.26 | 0.96 |
| Dioxide | | | | |
| Total | | | 131.66 | 100.00 |

*USP = United States Pharmacopeia; NF = National Formulary

TABLE 10
Formulation B:

| | | | | |
|---|---|---|---|---|
| | Qual- | | | |
| Component | ity* | Function | mg/dose | % w/w |

Pellet Core

| | | | | |
|---|---|---|---|---|
| Oxycodone | USP | Drug | 20.00 | 13.92 |
| hydrochloride | | Substance | | |
| Microcrystalline | USP | Filter/Diluent | 75.00 | 52.22 |
| Cellulose | | | | |
| Povidone (Kollidon 30) | USP | Filter/Diluent | 4.00 | 2.79 |
| Polyoxyl 35 Castor Oil | NF | Lubricant | 1.00 | 0.70 |
| Purified Water | USP | Process Aid | — | — |

Pellet Barrier Film Coat

| | | | | |
|---|---|---|---|---|
| Ammonio Methacrylate | NF | Film Forming | 3.09 | 2.15 |
| Copolymer, Type A (RL) | | Agent | | |
| Ammonio Methacrylate | NF | Film Forming | 12.36 | 8.61 |
| Copolymer, Type B (RS) | | Agent | | |
| Triethyl Citrate | NF | Plasticizer | 1.55 | 1.08 |
| Magnesium Stearate | NF | Antitacking | 3.00 | 2.09 |
| | | Agent | | |
| Isopropyl Alcohol | USP | Process Aid | — | — |
| Purified Water | USP | Process Aid | — | — |

Pellet Enteric Film Coat

| | | | | |
|---|---|---|---|---|
| Methacrylic Acid | NF | Film Forming | 13.91 | 9.68 |
| Copolymer Disp., | | Agent | | |
| Type C | | | | |
| Triethyl Citrate | NF | Plasticizer | 1.39 | 0.97 |
| Talc | NF | Antitacking | 6.95 | 4.84 |
| | | Agent | | |
| Isopropyl Alcohol | USP | Process Aid | — | — |
| Purified Water | USP | Process Aid | — | — |

Dusting Powder

| | | | | |
|---|---|---|---|---|
| Colloidal Silicon | NF | Lubricant | 1.38 | 0.96 |
| Dioxide | | | | |
| Total | | | 143.63 | 100.00 |

*USP = United States Pharmacopeia; NF = National Formulary

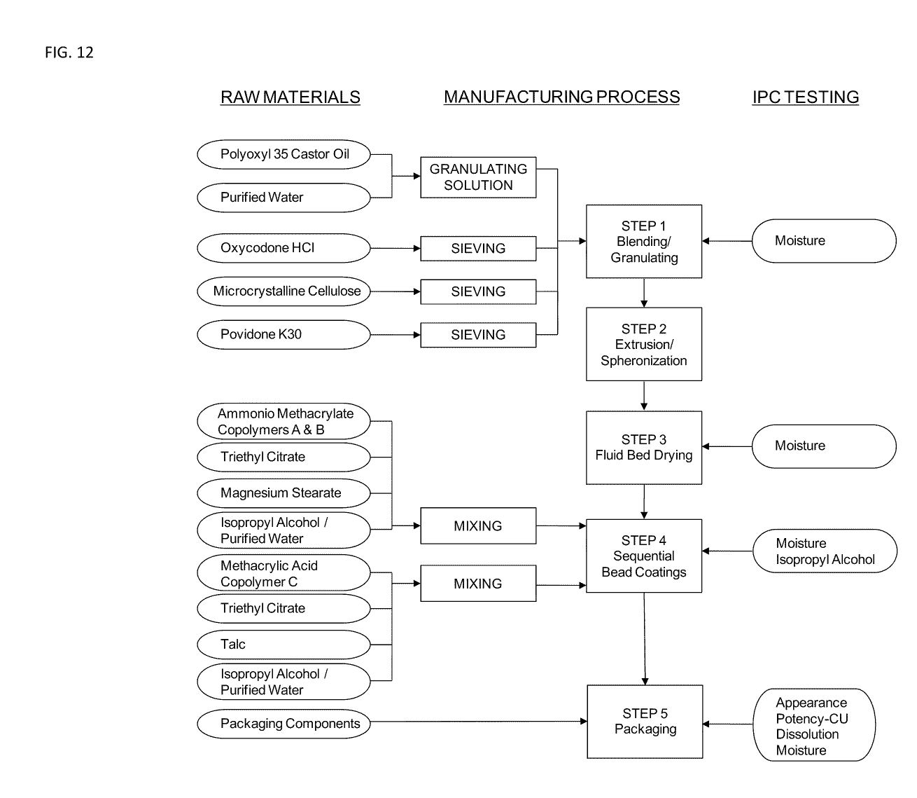

The manufacturing process of mixing the formulations is illustrated in the flow diagram of FIG. 12. To prepare the formulations, oxycodone hydrochloride, microcrystalline cellulose, and Povidone (Kollidon 30) were individually, manually screened through a # 20 mesh screen into a collecting container. The screened mix was transferred to a granulation bowl of a high shear granulator and dry mixed for three minutes.

A granulating solution comprising purified water mixed with Polyoxyl 35 Castor Oil was sprayed at a constant rate into the granulation bowl, mixing at low-speed-impeller or low-speed-chopper setting. The resulting granulation mixture was visually assessed continuously, and additional purified water was sprayed onto the mass as required.

The granulation mixture then underwent an extrusion-spheronization process using an extruder and plate spheronizer. The wet mass was uniformly extruded through a 0.8 mm screen into the marmurizing bowl where the extrudate was formed into appropriate sized pellets.

The pellets were dried using a Fluid Bed Dryer Granulator to a Loss on Drying (LOD) test target of ≦3%. To obtain the preferred fraction, the dried pellets were sieved through a #20 and #40 mesh size stainless steel screen into a double polyethylene-lined fiber drum for storage pending pellet spray coating.

The pellets then underwent spray coating using a Fluid Bed Dryer. In a stainless steel vessel, the coating components were mixed into an isopropyl alcohol/water solution using a pneumatic propeller mixer for at least one hour until a clear solution resulted. In a separate stainless steel vessel, the enteric coating solution was prepared by mixing the enteric coating components with a pneumatic mixer for at least one hour until a clear solution resulted. The polymer coating solutions were sprayed onto the pellets while continuously monitoring the spray conditions. The completed pellets were discharged into a double polyethylene-lined fiber drum for work-in-process storage pending lubrication.

The lubricated pellets were sieved through a # 18 and # 40 mesh size stainless steel screen to obtain the preferred fraction, and discharged into a double polyethylene-lined fiber drum for storage pending tablet blending.

### Example 4

### Pharmacokinetic Testing of Formulations A and B

Methods

A single-dose, three-period, three-sequence, three-treatment crossover study was conducted to compare the oxycodone pharmacokinetic profile human subjects orally administered Formulation A or B as described in Example 3, or with a Reference Formulation (MS Contin® 30 mg (morphine CR) co-administered with OxyContin® 20 mg (oxycodone CR)).

Each subject participated in a series of three periods, wherein each period was comprised of (i) pre-administration screening and check-in, (ii) administration of the formulation, and (iii) post-administration sample collection and follow-up. The subjects received a different formulation in each period, and were divided randomly to determine in which order the formulations were administered.

The pre-administration screening and check-in involved a physical examination and recordation of the subject's vital signs. Naltrexone (50 mg), an opioid antagonist, was administered 0.5 hours prior to administration. Blood samples were collected at 10 minutes and after 0.5, 1, 2, 3, 4, 5, 5.5, 6, 6.5, 7, 8, 10, 12, 14, 18, 21, 24, 48, and 72 hours post-dose of the formulation.

Morphine and oxycodone in the plasma of the blood samples were measured by liquid chromatography with tandem mass spectrometry (LC/MS/MS) methods that were validated across the following ranges:

Morphine 0.25-100 ng/mL

Oxycodone 50-50,000 pg/mL

Results

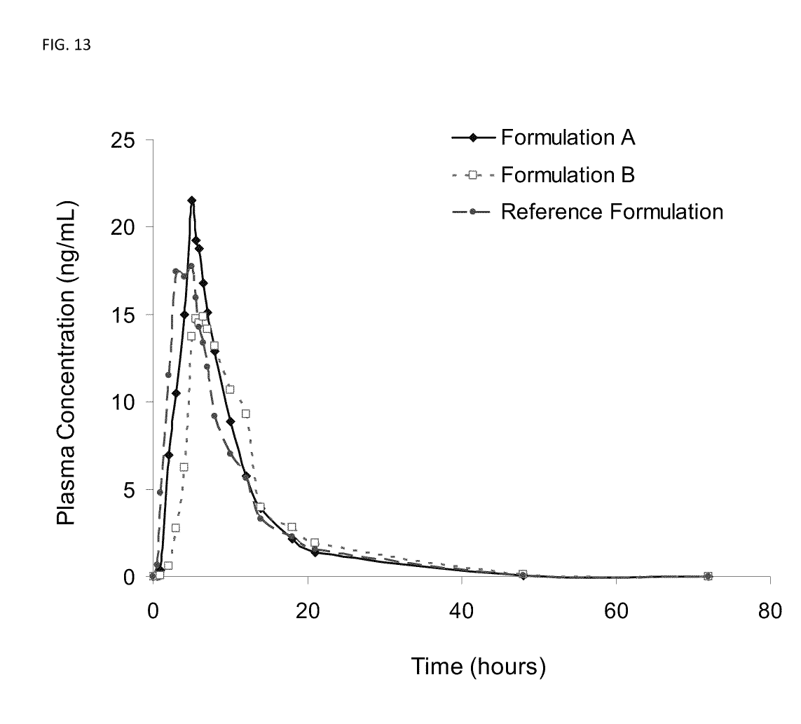

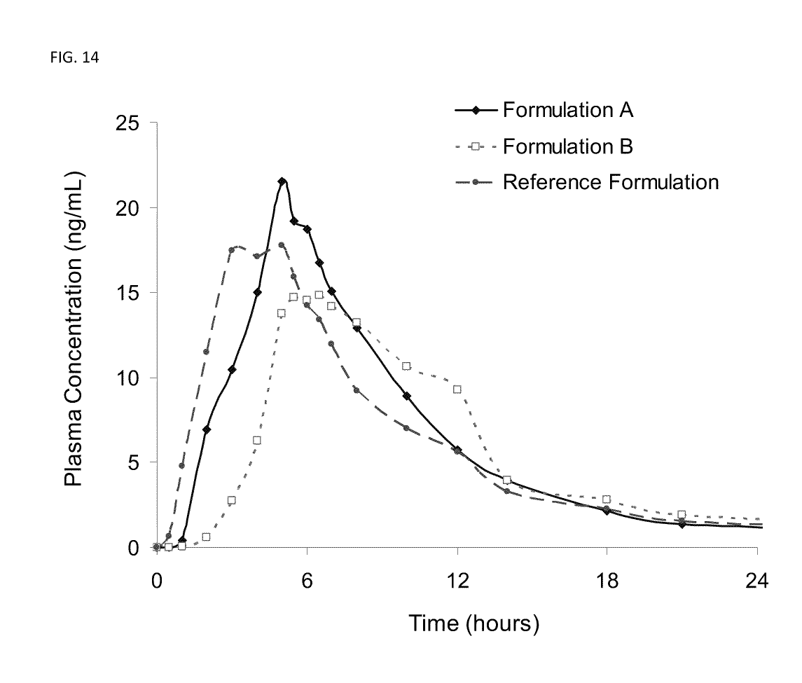

The mean plasma concentration of oxycodone at the sample collection timepoints is shown in FIG. 13 (through 72 hours) and FIG. 14 (the first 24 hours). As compared to the Reference Formulation, Formulation A resulted in higher plasma levels of oxycodone between 5 and 16 hours after treatment, although the plasma levels were generally lower thereafter. On other hand, Formulation B produced about the same or greater plasma levels of oxycodone as compared to the Reference Formulation at 6 hours after treatment and continuing through 48 hours. During this period, the plasma levels of oxycodone provided by Formulation B were, on average, 30% greater than the plasma levels provided by the Reference Formulation.

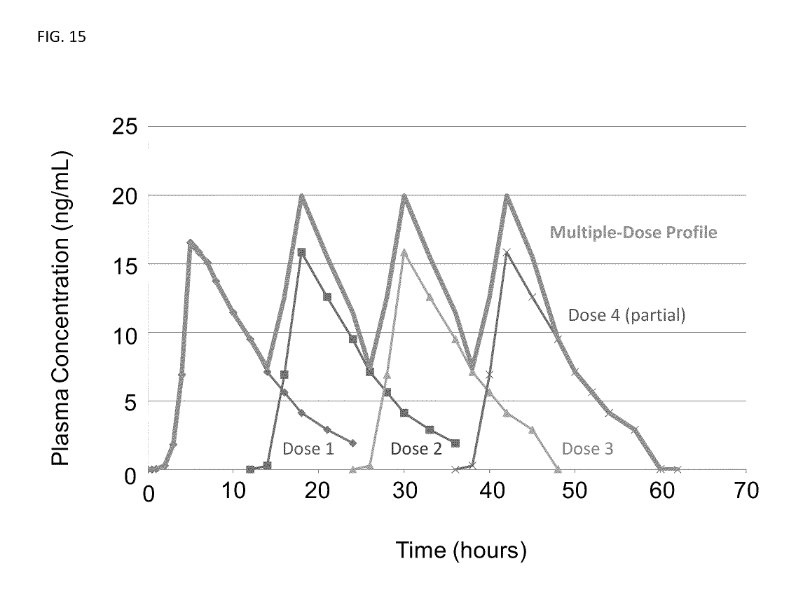

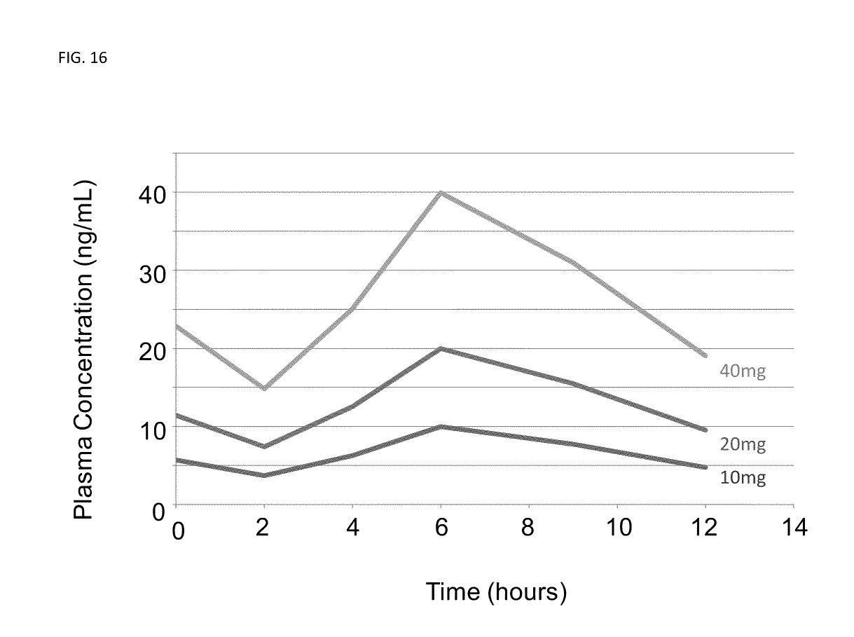

These data were used to project oxycodone plasma profiles that would result from administering multiple doses of Formulation B, as shown in FIGS. 15 and 16. FIG. 15 presents the oxycodone plasma profile through administration of 4 doses of Formulation B and indicates that, under this dosing regimen, oxycodone plasma levels can be maintained between about 7 and about 20 ng/mL.

FIG. 16 shows the oxycodone plasma profile that may result from different dosing strengths, and focuses on a single dose with the multiple dose regimen after the plasma levels achieve a steady-state; steady state is characterized by consistent peaks and troughs in the multiple dose plasma profile. FIG. 16 indicates that, at steady state, Cₘₐₓ will reflect the strength of the administered dose.

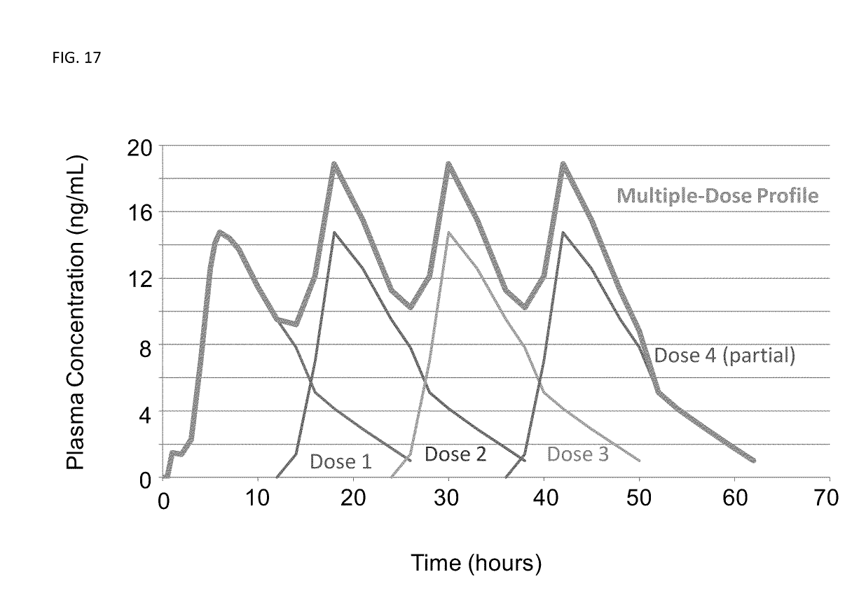

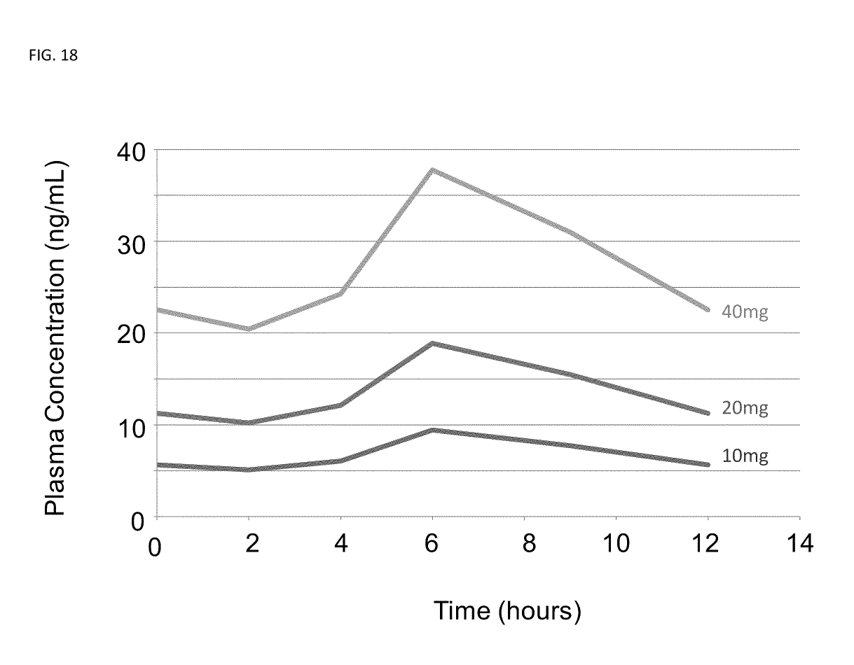

FIGS. 17 and 18 present projections of the oxycodone plasma profile for multiple doses of a formulation comprising a composite of an immediate release formulation (10%) and Formulation B (90%). FIG. 17 demonstrates the oxycodone plasma profile through administration of 4 doses of the composite formulation and indicates that, under this dosing regimen, oxycodone plasma levels can be maintained between about 10 and about 19 ng/mL.

FIG. 18 shows the oxycodone plasma profile that may result following administration of the composite formulation at different dosing strengths. FIG. 18 focuses on a single dose with the multiple dose regimen after the plasma levels achieve a steady-state, which is characterized by consistent peaks and troughs in the multiple dose plasma profile. The projection indicates that, at steady state, Cₘₐₓ will be less than the administered dose.

Comparisons of the oxycodone plasma profile of Formulation A to the Reference Formulation and the oxycodone plasma profile of Formulation B to the Reference Formulation are shown in Tables 11 and 12.

TABLE 11
Comparing Formulation A and the Reference Formulation

| | Formulation A | Reference Formulation |
|---|---|---|
| AUCₜ [pg · hr/mL] | 167077.87 ± 18761.51 | 194706.30 ± 41996.62 |
| Cₘₐₓ [pg/mL] | 24410.50 ± 4864.72 | 20525.70 ± 4520.50 |
| Tₘₐₓ [h] | 5.00 (2.00-6.00) | 3.00 (2.00-5.00) |

TABLE 12
Comparing Formulation A and the Reference Formulation

| | Formulation B | Reference Formulation |
|---|---|---|
| AUCₜ [pg · hr/mL] | 180846.58 ± 22868.36 | 194706.30 ± 41996.62 |
| Cₘₐₓ [pg/mL] | 16471.00 ± 3543.53 | 20525.70 ± 4520.50 |
| Tₘₐₓ [h] | 5.75 (5.00-12.0) | 3.00 (2.00-5.00) |

While AUCₜ of Formulations A and B were less than AUCₜ of the Reference Formulation, AUCₜ of Formulations A and B were within 14% and 7%, respectively. Also, Tₘₐₓ of both Formulations A and B were greater than Tₘₐₓ of the Reference Formulation, which was not expected.

### Example 5

### Immediate-Release Composition of Oxycodone with Controlled Release Mixture of Oxycodone-Morphine

An oral solid oral component tablet, comprising a core of 5.0 mg oxycodone hydrochloride and 5.0 mg morphine sulfate as active ingredients together with ammonio methacrylate copolymer, hypromellose, lactose, magnesium stereate, polyethylene glycol 400, povidone, sodium hydroxide, sorbic acid, stearyl alcohol, talc, titanium dioxide and triacetin, is prepared according to standard methods known in the art for preparation of tablets. The outside of the tablet is coated with a controlled release formulation comprising 10 mg of oxycodone hydrochloride and gelatin, hypromellose, maize starch, polyethylene glycol, polysorbate 80, red iron oxide, silicon dioxide, dodium laurel sulfate, sucrose, titanium dioxide and yellow iron oxide. The resulting tablet is administered to patients for the alleviation of pain and results in effective analgesia with no incidence of morphine-induced respiratory depression.

### Example 6

### General Procedure for Preparation of Controlled Release Formulations

The following manufacturing description is provided by way of example for the preparation of an controlled release, compressed tablet containing morphine sulfate and oxycodone hydrochloride.

### Preparation of Pellet Cores

The active drug substances (morphine sulfate and oxycodone hydrochloride), microcrystalline cellulose, USP and Povidone K30, NF were individually manually screened through a #20 mesh screen into a collecting container. The screened mix was transferred to the granulation bowl of a high shear granulator such as the PMA-25 or PMA-65 and dry mixed for 3 minutes.

A granulating solution consisting of a previously mixed solution of Purified Water, USP and Polyoxyl 35 Castor Oil, NF was sprayed at a constant rate into the granulation bowl and mixed at low speed impeller/low speed chopper setting. Granulation outcome was visually assessed on a continuous basis and additional Purified Water, USP was sprayed onto the mass if required. At the end of the granulation period, a sample was removed for an in process test for water content.

After sampling was completed, the granulation was discharged to the extrusion-spheronization process using a Luwa extruder and plate spheronizer or equivalent. The wet mass was uniformly extruded through a 0.8 mm screen into the marmurizing bowl where the extrudate was formed into appropriate sized pellets.

Fluid bed drying of the pellets was conducted using suitable process parameters with a GPCG-3, GPCG-5 or equivalent to a Loss on Drying (LOD) test target of ≦5%. The dried pellets were sieved to obtain the preferred fraction through a #20 and #40 mesh size stainless steel screen into a double PE-lined fiber drum for work-in-process storage pending pellet spray coating.

### Preparation of Modified Release Coated Beadlets

The ammonio methacrylate copolymers and triethyl citrate were mixed using a pneumatic propeller mixer into an isopropyl alcohol/water solution contained in a stainless steel vessel for at least one hour until a clear solution was obtained. Talc was then added to the vessel with continuous stirring. Fluid bed spray coating of the core pellets was conducted using suitable process parameters with a GPCG-5 Wurster fitted with a 1.0 mm spray nozzle.

### Preparation of Enteric Coated Beadlets

In a separate container, the enteric coating solution was prepared by mixing methacrylic acid copolymer and triethyl citrate with a pneumatic mixer in a stainless steel vessel for at least one hour. Talc was then added to the vessel with continuous stirring. The polymer coating solutions were successively sprayed at a constant rate to completion onto the beadlets while the spray conditions were continuously monitored. The enteric coated beadlets were discharged into a double polyethylene-lined fiber drum for work-in-process storage pending lubrication.

### Example 7

### Dissolution Testing Method for Controlled Release Formulations

The dissolution test method was designed to be used with an automated dissolution sampling station (e.g., Varian VK 8000). If such an instrument is not available, appropriate adjustments can be made in order to pull samples manually.

| | |
|---|---|
| Apparatus: | USP <711> Apparatus 2 (Paddles) |
| | Automated Dissolution Sampling Station |
| Vessel Size/Type: | About 1000 mL/clear glass, round-bottom vessel |
| Rotation Speed: | About 50 rpm throughout |
| Media and | Stage 1 (Acid Stage) from 0-2 hours: 750 mL of |
| Volume: | acidic Dissolution Medium A at 37.0 ± |
| | 0.5° C. for 2 hours |
| | Stage 2 (Buffer Stage) from 2-11 hours: 1000 mL |
| | at 37.0 ± 0.5° C., created by adding 250 |
| | mL of Dissolution Medium B and 20 mL |
| | Dissolution Medium A to the remnants of the media |
| | in the vessel from Stage 1. The Stage 2 media |
| | should have a pH of about 6.8 |
| Test Temperature: | About 37.0 ± 0.5° C. |
| Sinker: | Basket Sinker (0.46″ × 0.80″) 40 Mesh, |
| | 316-SS wire cloth |
| Pull Volume: | About 10 mL |
| Profile Time-points: | About 1, 2, 3, 4, 6, 9 and 11 hours |
| Media Replacement: | No |
| Sampling: | Automated |
| Filter Type/Size: | In-line 10-μm polyethylene full flow filter |

### Example 8

### Controlled Release Opioid Formulation Compositions

Following the procedure of Example 6, the following formulations were prepared:

TABLE 13
Modified Release Beadlets Formulations Using Ammonio
Methacrylate Copolymer Having RS/RL Ratio of 90/10.

| | Quantity per | % per |
|---|---|---|
| Component | Unit (mg) | Unit (w/w) |
| Morphine Sulfate + Oxycodone HCl | 40 | 62.5 |
| Core Pellets | | |
| Ammonio Methacrylate copolymer | 16.3 | 25.5 |
| Type B (RS PO) | | |
| Ammonio Methacrylate copolymer | 1.8 | 2.8 |
| Type A (RL PO) | | |
| Triethyl Citrate NF/EP | 2.3 | 3.5 |
| Talc (197 Grade) USP/EP/JP | 3.6 | 5.6 |

| | |
|---|---|
| Water purified | Removed by evaporation |
| | during the coating process |

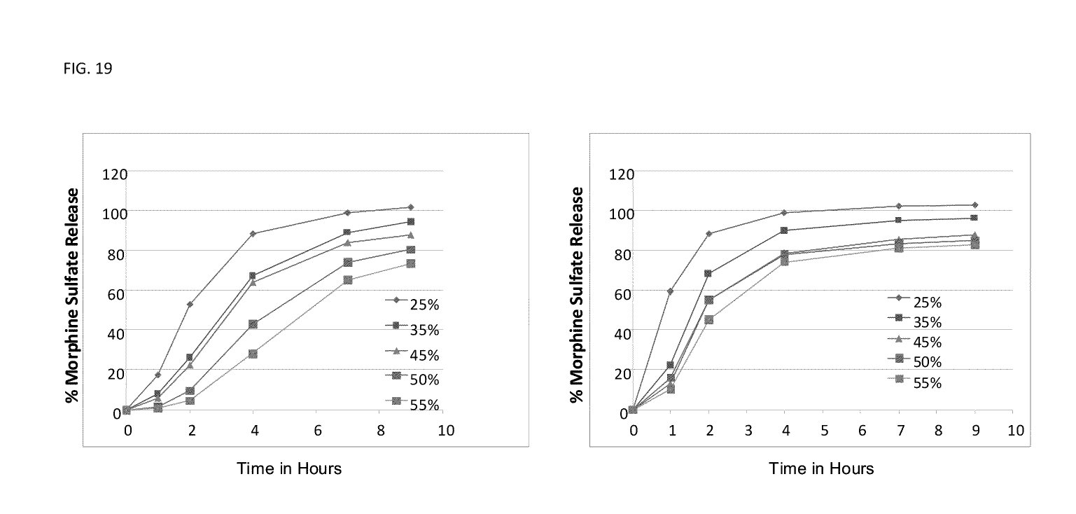

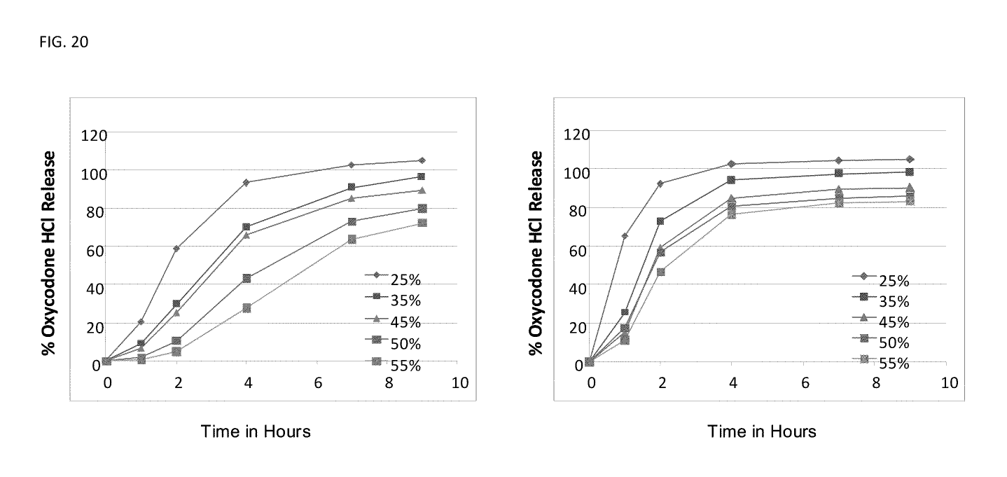

Various formulations were prepared having different % coating levels (e.g., 25%, 35%, 45%, 50% and 55%) of the ammonio methacrylate RS/RL polymers. FIGS. 19(a) and 20(a) provide representative dissolution profiles for morphine sulfate and oxycodone hydrochloride, respectively.

TABLE 14
Tablet Formulations Using Morphine/Oxycodone
Enteric Coated/Modified Release Beadlets.

| | Quantity per | % per |
|---|---|---|
| Component | Unit (mg) | Unit (w/w) |
| Morphine Sulfate + Oxycodone HCl | 20 | 20 |
| Modified Release Beadlets (RS/RL = | | |
| 90:10 + Enteric Coating) | | |
| Microcrystalline Cellulose PH101 | 73.1 | 73.1 |
| Granulated | | |
| Povidone K30 | 6.4 | 6.4 |
| Magnesium Stearate 5712 | 0.5 | 0.5 |

| | |
|---|---|
| 8% w/w Povidone solution | Water removed by |
| (used for granulation) | evaporation post process. |

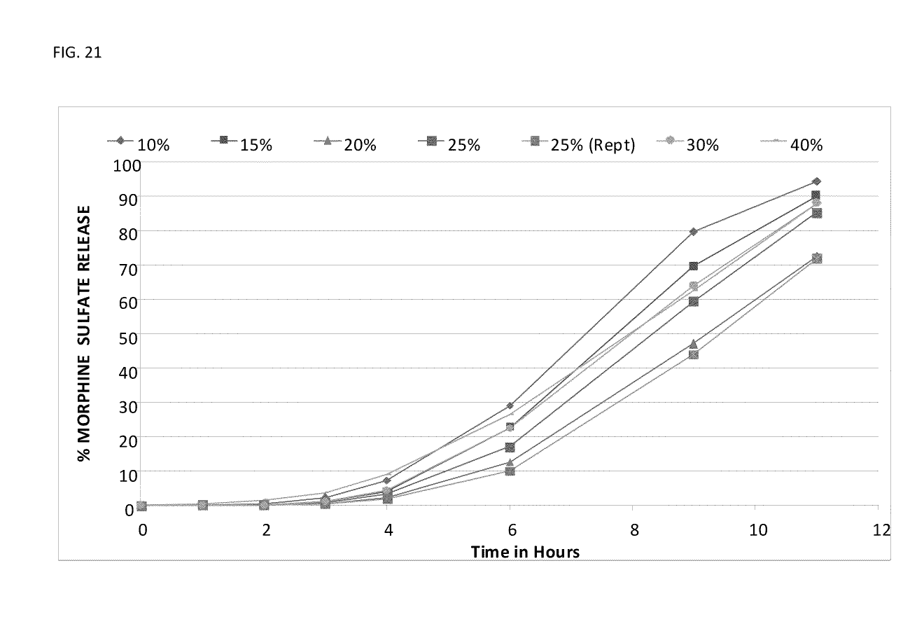

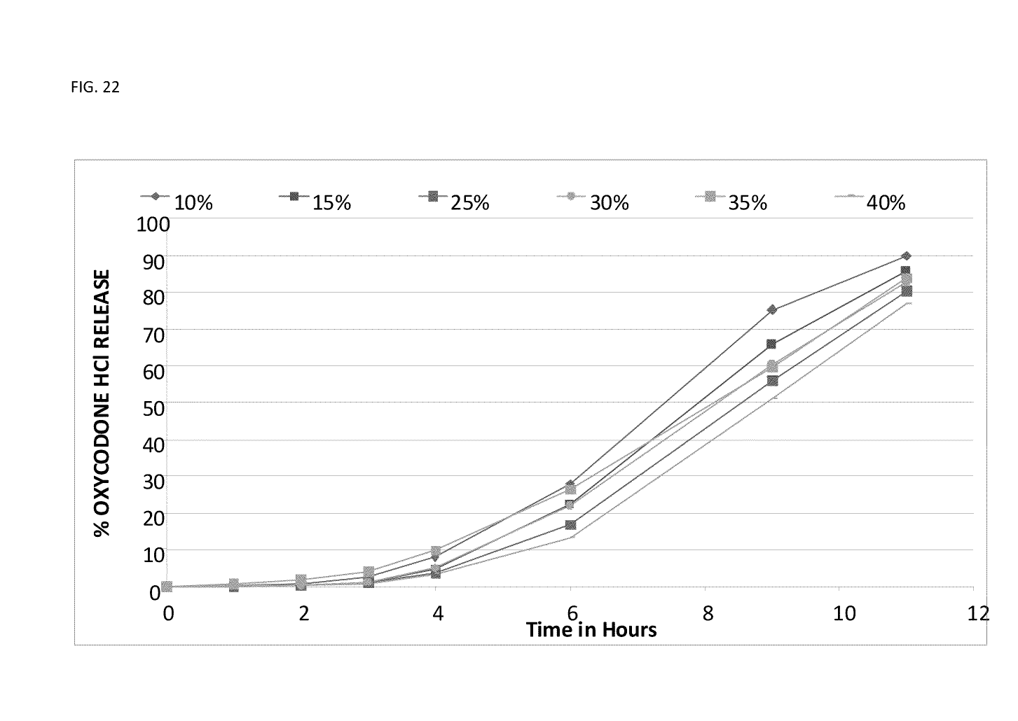

Various tablet formulations were prepared having different % enteric coating levels (e.g., 10%, 15%, 20%, 25%, 30% and 40%). FIGS. 21 and 22 provide representative dissolution profiles for morphine sulfate and oxycodone hydrochloride, respectively.

### Example 9

### Dissolution Testing of Various % Modified Release Coating Levels and Enteric Coating Levels

Two lots (˜3 kg) of morphine sulfate/oxycodone (3:2 by weight ratio) core pellets were coated using RS/RL polymer ratios of 90/10 (Lot 1, see Table 13) and 80/20 (Lot 2). Each lot was coated with different coating levels (25%, 35%, 45%, 50% and 55%) and samples were collected during the coating process. Dissolution testing (FIGS. 19 and 20) was performed on Lots 1 and 2 at the different coating levels.

In addition, coated pellets obtained from Lot 1 (at a 50 RS/RL coating level) were subjected to enteric coating at different % coating levels (10%, 15%, 25%, 30% and 40%) to produce enteric coated tablets and dissolution testing was performed (FIGS. 21 and 22).

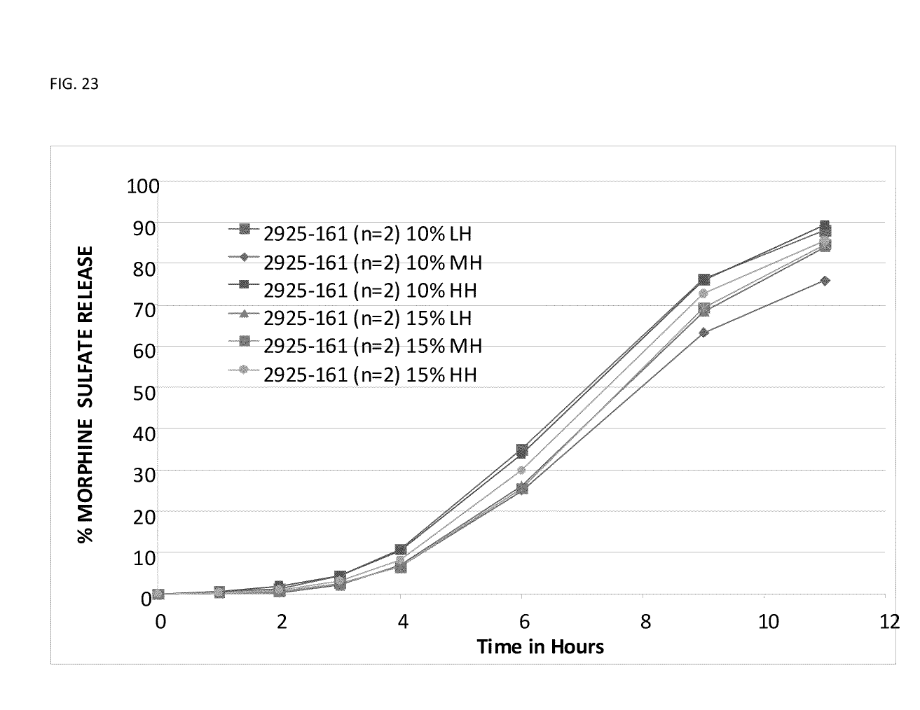

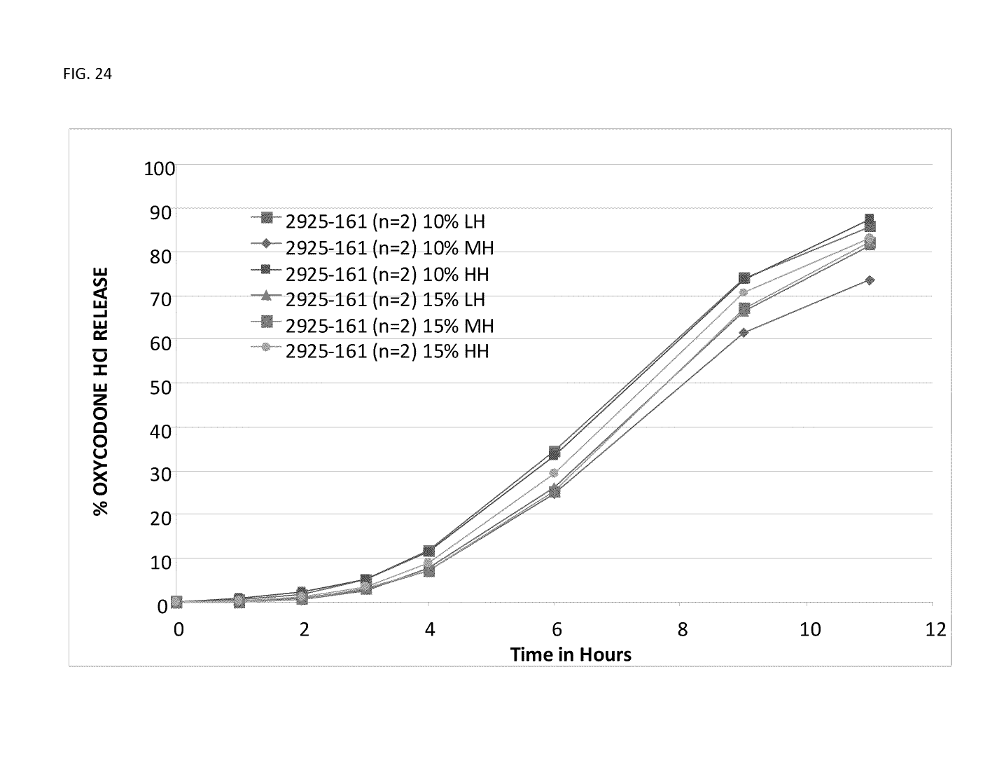

Enteric coated tablet lots (using 10% and 15% enteric coat) were also analyzed for dissolution as a function of tablet hardness (low, medium or high) to determine the resistance of the tablets to various compression levels (FIGS. 23 and 24).

A summary of the dissolution testing is provided in Table 15.

TABLE 15
Dissolution Testing Experiments.

| Test Performed | Batch # | Stage | Coating Level % |
|---|---|---|---|
| Dissolution | 2925-069 | Modified Release | 25 |
| | (RS/RL | Coated Beads | 35 |
| | 80/20) | | 45 |
| | | | 50 |
| | | | 55 |
| Dissolution | 2925-076 | Modified Release | 25 |
| | (RS/RL | Coated Beads | 35 |
| | 90/10) | | 45 |
| | | | 50 |
| | | | 55 |
| Dissolution | 2925-115 | Enteric Coated Tablets | 10 |
| | (RS/RL | (50% Modified Release | 15 |
| | 90/10) | Coated Beads) | 25 |
| | | | 30 |
| | | | 40 |
| Test Performed | Batch # | Stage | Tablet Hardness |
| Dissolution | 2925-161 | Tablet Compression | Low Hardness |
| | (RS/RL ) | (10% Enteric Coated | Mid Hardness |
| | 90/10 | Tablets) | High Hardness |
| Dissolution | 2925-161 | Tablet Compression | Low Hardness |
| | (RS/RL | (15% Enteric Coated | Mid Hardness |
| | 90/10) | Tablets) | High Hardness |

FIGS. 19 and 20 show the versatility of modified release core beadlets at various % coating levels in obtaining the dissolution profile of interest. A full spectrum of dissolution profiles allows for the targeting of specific in vivo pharmacokinetic plasma levels and the determination of in vitro to in vivo correlations.

FIGS. 21 and 22 also show the versatility of enteric coated, modified release core beadlets at various % enteric coating levels in obtaining the dissolution profile of interest. Once again, a full spectrum of dissolution profiles allows for the targeting of specific in vivo pharmacokinetic plasma levels and the determination of in vitro to in vivo correlations.

FIGS. 23 and 24 show the effect of compression forces on tablets that contain enteric coated beadlets comprising a modified release coated pellet of morphine sulfate and oxycodone hydrochloride. It is generally known that a high compression force can significantly reduce the dissolution of tablets, especially when coating polymers are employed that are known to be brittle, such as with ammonio methacrylate copolymer Type A and B. FIGS. 23 and 24 demonstrate that a low or high compression force does not affect the dissolution of tablets. This result is unexpected and demonstrates the resilience of the formulation/coatings to compression forces.

### Example 10

### Controlled Release Opioid Formulation Compositions

Following the procedure of Example 6, the following formulations were prepared:

TABLE 16
Tablet Formulations of Modified Release Beadlets (RS/RL)
with/without Enteric Coating (Eudragit L100-55 Type C).

| | Modified Release | | Modified Release | | | |
|---|---|---|---|---|---|---|
| | RS/RL 85/15 | | RS/RL 80/20 | | | |
| | Modified Release Coating Level % | | | | | |
| | 10% | 15% | 10% | 20% | 10% | 20% |
| Component | % w/w | | | | | |
| Oxycodone Hydrochloride | 18.18 | 17.39 | 18.09 | 16.58 | 15.19 | 13.92 |
| Cellulose Microcrystalline | 68.18 | 65.22 | 63.32 | 58.04 | 56.96 | 52.22 |
| Povidone K30 | 3.64 | 3.48 | — | — | 3.04 | 2.79 |
| Hypromellose | — | — | 8.14 | 7.46 | — | — |
| (Methocel E15 Premium LV) | | | | | | |
| Polyoxyl 35 Castor Oil | 0.91 | 0.87 | 0.90 | 0.83 | 0.76 | 0.70 |
| Ammonio Methacrylate | 4.68 | 6.71 | 5.59 | 10.25 | 4.70 | 8.61 |
| Copolymer Type B (RS PO) | | | | | | |
| Ammonio Methacrylate | 0.83 | 1.18 | 1.40 | 2.56 | 1.17 | 2.15 |
| Copolymer Type A (RL PO) | | | | | | |
| Triethyl Citrate NF/EP | 0.86 | 1.24 | 0.70 | 1.28 | 0.59 | 1.08 |
| Magnesium Stearate 5712 | 2.73 | 3.91 | 1.36 | 2.49 | 1.14 | 2.09 |
| Enteric Coating | — | — | — | — | 9.68 | 9.68 |
| Eudragit L 100-55 Type C | | | | | | |
| Triethyl Citrate NF/EP | — | — | — | — | 0.97 | 0.97 |
| Talc (197 Grade) USP/EP/JP | — | — | 0.5 | 0.5 | 4.84 | 4.84 |
| Silicon Dioxide Colloidal | — | — | — | — | 0.96 | 0.96 |
| Total | 100 | 100 | 100 | 100 | 100 | 100 |

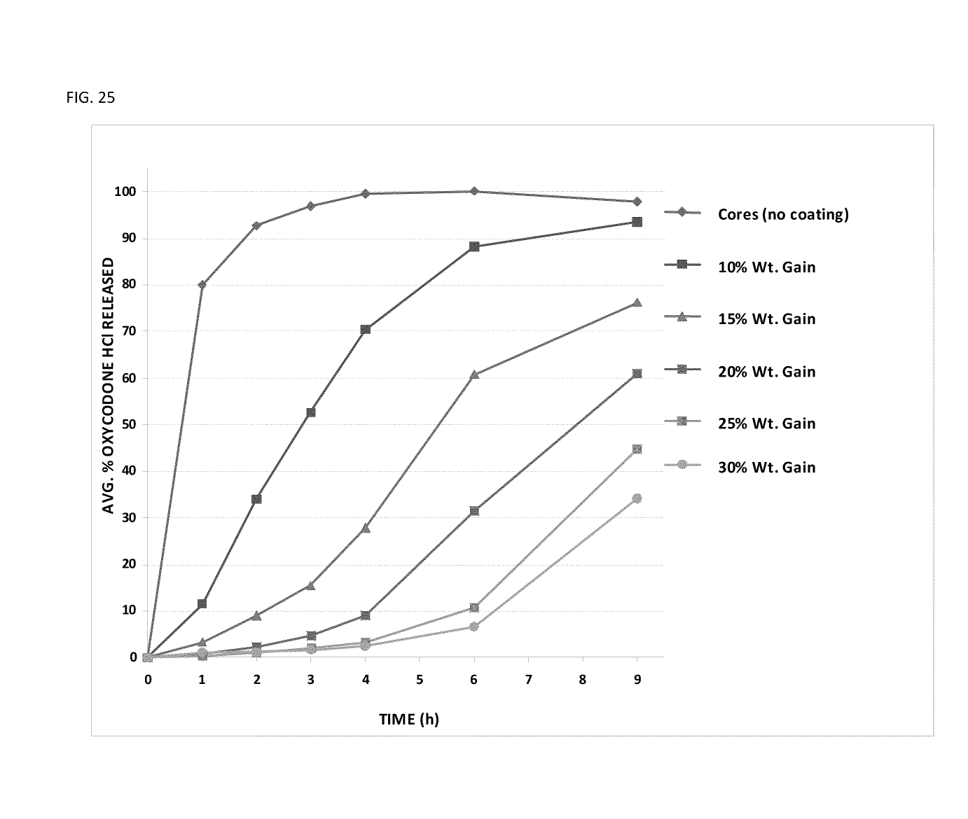

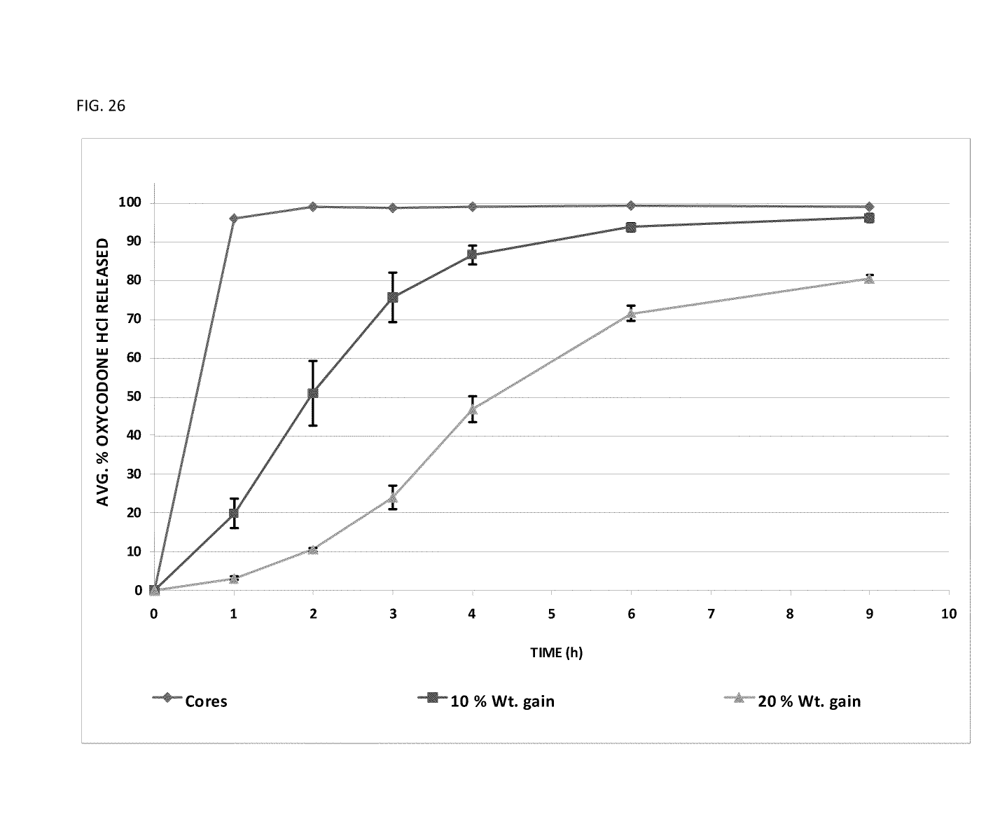

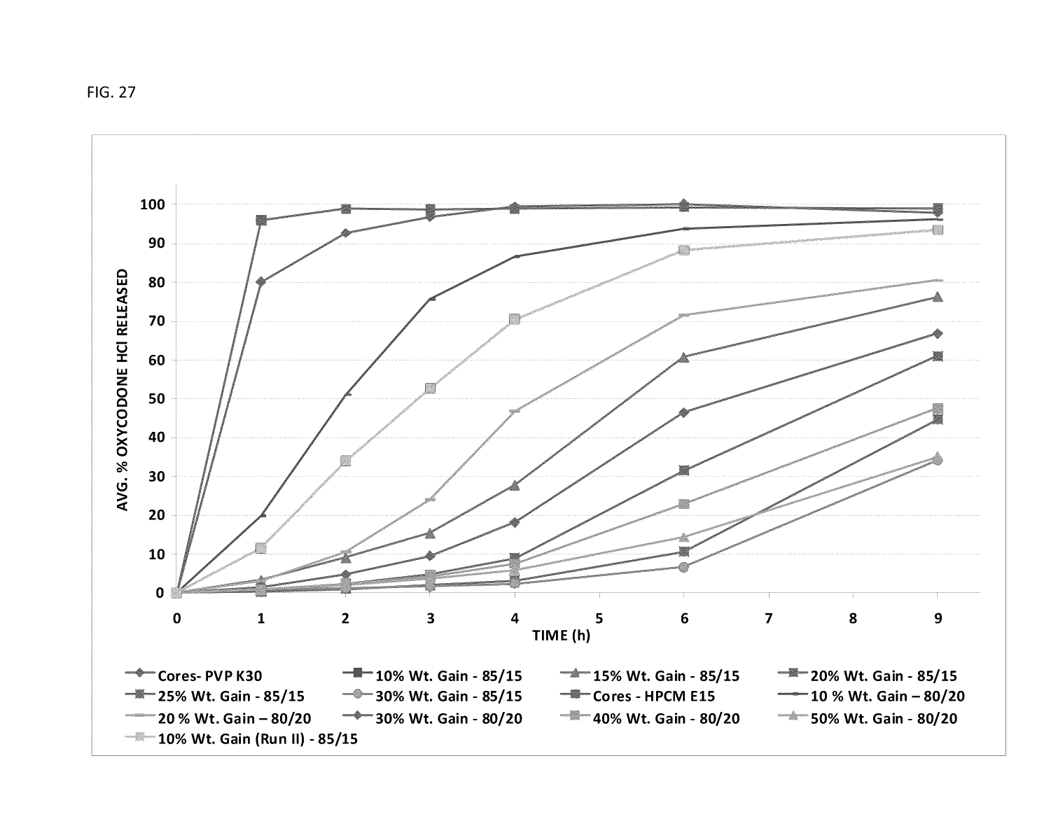

FIGS. 25-27 provide representative dissolution profiles for morphine sulfate and oxycodone hydrochloride, respectively, for the formulations provided in Table 16. These figures show the versatility of modified release core beadlets at various % coating levels in obtaining the dissolution profile of interest. Enteric coated beadlet formulations are also provided that allow for a full spectrum of dissolution profiles to be achieved.

It should be understood, of course, that the foregoing relates only to certain disclosed embodiments of the present invention and that numerous modifications or alterations may be made therein without departing from the spirit and scope of the invention as set forth in the appended claims.

What is claimed is:

1. A pharmaceutical formulation for treatment of pain in a human, comprising one or more opioid components, wherein:
(a) the one or more opioid components comprise one or more release profiles;
(b) at least one of the opioid components is a controlled release opioid component comprising an opioid, wherein the opioid is oxycodone or a salt thereof;
wherein the pharmaceutical formulation provides a time to maximum oxycodone, or a salt thereof, plasma concentration (Tₘₐₓ) of about 4.5 to about 8 hours after repeated administration.

2. The pharmaceutical formulation of claim 1, wherein the controlled release opioid component further comprises one or more additional opioids selected from the group consisting of morphine, codeine, hydromorphone, hydrocodone, dihydrocodeine, dihydromorphine, oxymorphone, mixtures thereof, and salts thereof.

3. The pharmaceutical formulation of claim 1, wherein the formulation, when containing a total dose of about 2 mg of oxycodone, or a salt thereof, provides an area-under-the-curve for between about 0 and about 24 hours (AUC₂₄) of about 14.7 ng·hr/mL to about 23.0 ng·hr/mL after single administration.

4. The pharmaceutical formulation of claim 3, formulated for a total dose of oxycodone, or a salt thereof, that is different from about 2 mg and has an AUC₂₄ that is proportional to the 2 mg AUC₂₄.

5. The pharmaceutical formulation of claim 1, wherein the formulation, when containing a total dose of about 2 mg of oxycodone, or a salt thereof, provides a mean maximum oxycodone, or a salt thereof, plasma concentration (Cₘₐₓ) of about 1 to about 3 ng/mL after repeated administration through steady-state conditions.

6. The pharmaceutical formulation of claim 5, formulated for a total dose of oxycodone, or a salt thereof, that is different from about 2 mg and has a Cₘₐₓ that is proportional to the 2 mg Cₘₐₓ.

7. The pharmaceutical formulation of claim 1, wherein the formulation, when containing a total dose of about 5 mg of oxycodone, or a salt thereof, provides an area-under-the-curve for between about 0 and about 24 hours (AUC₂₄) of about 40.2 ng·hr/mL to about 62.8 ng·hr/mL after single administration.

8. The pharmaceutical formulation of claim 7, formulated for a total dose of oxycodone, or a salt thereof, that is different from about 5 mg and has an AUC₂₄ that is proportional to the 5 mg AUC₂₄.

9. The pharmaceutical formulation of claim 1, wherein the formulation, when containing a total dose of about 5 mg of oxycodone, or a salt thereof, provides a mean maximum oxycodone, or a salt thereof, plasma concentration (Cₘₐₓ) of about 3 to about 7 ng/mL after repeated administration through steady-state conditions.

10. The pharmaceutical formulation of claim 9, formulated for a total dose of oxycodone, or a salt thereof, that is different from about 5 mg and has a Cₘₐₓ that is proportional to the 5 mg Cₘₐₓ.

11. The pharmaceutical formulation of claim 1, wherein the formulation, when containing a total dose of about 10 mg of oxycodone, or a salt thereof, provides an area-under-the-curve for between about 0 and about 24 hours (AUC₂₄) of about 80.5 ng·hr/mL to about 125.9 ng·hr/mL after single administration.

12. The pharmaceutical formulation of claim 11, formulated for a total dose of oxycodone, or a salt thereof, that is different from about 10 mg and has an AUC₂₄ that is proportional to the 10 mg AUC₂₄.

13. The pharmaceutical formulation of claim 1, wherein the formulation, when containing a total dose of about 10 mg of oxycodone, or a salt thereof, provides a mean maximum oxycodone, or a salt thereof, plasma concentration (Cₘₐₓ) of about 5 to about 15 ng/mL after repeated administration through steady-state conditions.

14. The pharmaceutical formulation of claim 13, formulated for a total dose of oxycodone, or a salt thereof, that is different from about 10 mg and has a Cₘₐₓ that is proportional to the 10 mg Cₘₐₓ.

15. The pharmaceutical formulation of claim 1, wherein the formulation, when containing a total dose of about 20 mg of oxycodone, or a salt thereof, provides an area-under-the-curve for between about 0 and about 24 hours (AUC₂₄) of about 166.0 ng·hr/mL to about 259.3 ng·hr/mL after single administration.

16. The pharmaceutical formulation of claim 15, formulated for a total dose of oxycodone, or a salt thereof, that is different from about 20 mg and has an AUC₂₄ that is proportional to the 20 mg AUC₂₄.

17. The pharmaceutical formulation of claim 1, wherein the formulation, when containing a total dose of about 20 mg of oxycodone, or a salt thereof, provides a mean maximum oxycodone, or a salt thereof, plasma concentration (Cₘₐₓ) of about 10 to about 30 ng/mL after repeated administration through steady-state conditions.

18. The pharmaceutical formulation of claim 17, formulated for a total dose of oxycodone, or a salt thereof, that is different from about 20 mg and has a Cₘₐₓ that is proportional to the 20 mg Cₘₐₓ.

19. The pharmaceutical formulation of claim 1, wherein the formulation, when containing a total dose of about 40 mg of oxycodone, or a salt thereof, provides an area-under-the-curve for between about 0 and about 24 hours (AUC₂₄) of about 338.5 ng·hr/mL to about 528.9 ng·hr/mL after single administration.

20. The pharmaceutical formulation of claim 19, formulated for a total dose of oxycodone, or a salt thereof, that is different from about 40 mg and has an AUC₂₄ that is proportional to the 40 mg AUC₂₄.

21. The pharmaceutical formulation of claim 1, wherein the formulation, when containing a total dose of about 40 mg of oxycodone, or a salt thereof, provides a mean maximum oxycodone, or a salt thereof, plasma concentration (Cₘₐₓ) of about 25 to about 55 ng/mL after repeated administration through steady-state conditions.

22. The pharmaceutical formulation of claim 21, formulated for a total dose of oxycodone, or a salt thereof, that is different from about 40 mg and has a Cₘₐₓ that is proportional to the 40 mg Cₘₐₓ.

23. The pharmaceutical formulation of claim 1, wherein the formulation, when containing a total dose of about 80 mg of oxycodone, or a salt thereof, provides an area-under-the-curve for between about 0 and about 24 hours (AUC₂₄) of about 868.4 ng·hr/mL to about 1356.9 ng·hr/mL after single administration.

24. The pharmaceutical formulation of claim 23, formulated for a total dose of oxycodone, or a salt thereof, that is different from about 80 mg and has an AUC₂₄ that is proportional to the 80 mg AUC₂₄.

25. The pharmaceutical formulation of claim 1, wherein the formulation, when containing a total dose of about 80 mg of oxycodone, or a salt thereof, provides a mean maximum oxycodone, or a salt thereof, lasma concentration (Cₘₐₓ) of about 50 to about 110 ng/mL after repeated administration through steady-state conditions.

26. The pharmaceutical formulation of claim 25, formulated for a total dose of oxycodone, or a salt thereof, that is different from about 80 mg and has a Cₘₐₓ that is proportional to the 80 mg Cₘₐₓ.

27. The pharmaceutical formulation of claim 1, comprising a second controlled release opioid component.

28. The pharmaceutical formulation of claim 27, wherein the second controlled release opioid component comprises an opioid selected from the group consisting of morphine, codeine, hydromorphone, hydrocodone, oxycodone, dihydrocodeine, dihydromorphine, oxymorphone, mixtures thereof, and salts thereof.

29. The pharmaceutical formulation of claim 1, comprising an immediate-release opioid component.

30. The pharmaceutical formulation of claim 29, where the immediate-release opioid component comprises an opioid selected from the group consisting of morphine, codeine, hydromorphone, hydrocodone, oxycodone, dihydrocodeine, dihydromorphine, oxymorphone, mixtures thereof, and salts thereof.

31. The pharmaceutical formulation of claim 30, wherein the opioid in the immediate-release opioid component is morphine or a salt thereof.

32. The pharmaceutical formulation of claim 31, wherein the total morphine, or salt thereof, and the total oxycodone, or salt thereof, in the formulation are in a ratio of about 3:2, morphine or salt thereof to oxycodone or salt thereof, by weight.

33. The pharmaceutical formulation of claim 1, comprising a second opioid component and a third opioid component, wherein:
(a) the second opioid component is an immediate-release opioid component and comprises an opioid having kappa agonist activity; and
(b) the third opioid component is a controlled release opioid component and comprises an opioid having mu agonist activity.

34. The pharmaceutical formulation of claim 33, wherein the opioid having kappa agonist activity is oxycodone or a salt thereof.

35. The pharmaceutical formulation of claim 33, wherein the opioid having mu agonist activity is morphine or a salt thereof.

36. The pharmaceutical formulation of claim 1, wherein the controlled release opioid component comprises morphine or a salt thereof.

37. The pharmaceutical formulation of claim 36, wherein the controlled release opioid component comprises morphine or salt thereof and oxycodone or salt thereof in an amount of about 3:2 by weight.

38. The pharmaceutical formulation of claim 1, wherein dissolution of the formulation releases about 0 to about 20% of the oxycodone, or a salt thereof, after two hours as measured in a USP type I apparatus at about 37° C. in water at about 50 rpm.

39. The pharmaceutical formulation of claim 1, wherein dissolution of the formulation releases about 15 to about 60% of the oxycodone, or a salt thereof, after four hours as measured in a USP type I apparatus at about 37° C. in water at about 50 rpm.

40. The pharmaceutical formulation of claim 1, wherein dissolution of the formulation releases about 25 to about 80% of the oxycodone, or a salt thereof, after six hours as measured in a USP type I apparatus at about 37° C. in water at about 50 rpm.

41. The pharmaceutical formulation of claim 1, wherein dissolution of the formulation releases about 35 to about 85% of the oxycodone, or a salt thereof, after eight hours as measured in a USP type I apparatus at about 37° C. in water at about 50 rpm.

42. The pharmaceutical formulation of claim 1, wherein dissolution of the formulation releases about 45 to about 95% of the oxycodone, or a salt thereof, after ten hours as measured in a USP type I apparatus at about 37° C. in water at about 50 rpm.

43. The pharmaceutical formulation of claim 1, wherein dissolution of the formulation releases about 60 to about 100% of the oxycodone, or a salt thereof, after twelve hours as measured in a USP type I apparatus at about 37° C. in water at about 50 rpm.

44. A method of controlling release of one or more compounds having opioid receptor agonist activity for absorption in a human, wherein the method comprises administering a pharmaceutical formulation comprising one or more components, wherein:
(a) the one or more opioid components comprise one or more release profiles;
(b) at least one of the opioid components is a controlled release opioid component comprising an opioid, wherein the opioid is oxycodone or a salt thereof;
wherein the pharmaceutical formulation provides a time to maximum oxycodone, or a salt thereof, plasma concentration (Tₘₐₓ) of about 4.5 to about 8 hours after repeated administration.

45. A method of treating pain in a human, comprising administering a pharmaceutical formulation comprising one or more components, wherein:
(a) the one or more opioid components comprise one or more release profiles;
(b) at least one of the opioid components is a controlled release opioid component comprising an opioid, wherein the opioid is oxycodone or a salt thereof;
wherein the pharmaceutical formulation provides a time to maximum oxycodone, or a salt thereof, plasma concentration (Tₘₐₓ) of about 4.5 to about 8 hours after repeated administration.
